(19) United States
(12) Patent Application Publication
(10) Pub. No.: US 2019/0183436 A1
(43) Pub. Date: Jun. 20, 2019

(54) INFORMATION PROCESSING DEVICE, INFORMATION PROCESSING SYSTEM, AND INFORMATION PROCESSING METHOD

(71) Applicant: Sony Corporation, Tokyo (JP)

(72) Inventors: Masakazu Yajima, Kanagawa (JP); Chisako Kajihara, Tokyo (JP); Hiromitsu Komatsu, Kanagawa (JP); Junya Matsui, Kanagawa (JP); Hideo Niikura, Tokyo (JP)

(73) Assignee: Sony Corporation, Tokyo (JP)

(21) Appl. No.: 16/093,281

(22) PCT Filed: Mar. 9, 2017
(86) PCT No.: PCT/JP2017/009361

§ 371 Date: Oct. 12, 2018

(30) Foreign Application Priority Data
Apr. 21, 2016 (JP) 2016-085250

(51) Int. Cl. A61B 5/00; A01K 29/00; A61B 10/00; A61B 5/11; H04Q 9/00

(52) U.S. Cl. CPC A61B 5/7465; H04W 4/38; A61B 10/0012; A61B 5/7275; A61B 5/7267; A61B 5/7435; A61B 5/7475; A61B 5/7425; A61B 5/1118; A61B 5/4866; A61B 5/002; A61B 5/0022; H04Q 9/00; A61B 2503/40; H04Q 2209/826; A01K 29/005

(57) ABSTRACT

[Solution] An information processing device, an information processing system, and an information processing method which can determine an estrus period of individual livestock while viewing a transition of the state quantity showing a characteristic change in the estrus period of livestock, and execute contact with a veterinarian and accumulation of information of the estrus period by an operation of causing the estrus period of livestock to be notified to a veterinarian when the estrus period has arrived.

[Solution] The information processing device includes: a display information control unit configured to cause a transition of a state quantity showing a characteristic change in an estrus period of livestock to be displayed on a display unit of a breeder-side information terminal; and a notification control unit configured to execute, in accordance with a notification request to cause estrus of the livestock to be notified to a veterinarian-side information terminal on a basis of an operation of the breeder-side information terminal, both notification processing to the veterinarian-side information terminal and registration processing of registering time information indicating a time at which the notification request is made in a database as estrus history of the livestock.

### TECHNICAL FIELD

The present disclosure relates to an information processing device, an information processing system, and an information processing method.

### BACKGROUND ART

It is important to know an estrus period in order to efficiently breed livestock. By ascertaining an estrus period, fertilization of livestock can be performed at an appropriate time, and it can contribute to preventing a conception rate from decreasing due to loss of opportunities for breeding. A breeder such as a rancher has determined estrus of livestock by carefully observing a state of individual livestock. However, as the number of livestock becomes large with increases in the scale of animal industry, a burden on the breeder increases, and thus it is desirable to efficiently manage the estrus period of livestock. For this reason, a technology in which detectors for measuring individual information of livestock are attached to the livestock, and a system determines estrus on the basis of measurement data sent from the detectors has been proposed.

For example, the following Patent Literature 1 discloses an estrus period information notification system for livestock which includes a livestock biological information measurement unit that is anchored in at least the vagina of a livestock animal to continuously measure and output biological information showing chronological features in the estrus period of the animal, a data relay unit that loads measurement data from the livestock biological information measurement unit on a communication line, an estrus period determination unit that continuously determines whether or not the biological information exhibits chronological features in the estrus period of the livestock on the basis of the measurement data sent on the communication line, and a notification unit that notifies an administrator of livestock immediately when it is determined that the biological information shows chronological features in the estrus period of the livestock.

### CITATION LIST

### Patent Literature

Patent Literature 1: JP 2002-159233 A

### DISCLOSURE OF INVENTION

### Technical Problem

Here, for example, the estrus period of dairy cattle comes in a 21-day cycle on average and lasts for 15 to 16 hours. However, there are individual differences in estrus cycle and duration time. For example, the estrus cycle may have an error of one week before or after. In addition, in a case in which the estrus time is short, it may last only about 5 hours in some cases. Therefore, accumulation of actual data of individual dairy cattle by a breeder determining the estrus of individual dairy cattle is effective to know individual differences. Data management of the estrus of individual dairy cattle has often been performed manually by a breeder, a veterinarian, or the like up to now, and data management efficiency is desired.

In addition, in a case in which livestock is in an estrus state, it is necessary to contact a veterinarian and perform fertilization processing. Since estrus itself is not abnormal, for example, even in a case in which estrus is seen at night, contact with a veterinarian is often performed the next day. For this reason, a breeder may forget to contact a veterinarian, and the veterinarian may need to prepare on the same day after receiving contact.

In view of this, in the present disclosure, novel and improved information processing device, information processing system, and information processing method which can determine estrus of individual livestock while viewing a transition of the state quantity showing a characteristic change in the estrus period of livestock, and execute contact with a veterinarian and accumulation of information of the estrus period are presented.

### Solution to Problem

According to the present disclosure, there is provided an information processing device including: a display information control unit configured to cause a transition of a state quantity showing a characteristic change in an estrus period of livestock to be displayed on a display unit of a breeder-side information terminal; and a notification control unit configured to execute, in accordance with a notification request to cause estrus of the livestock to be notified to a veterinarian-side information terminal on a basis of an operation of the breeder-side information terminal, both notification processing to the veterinarian-side information terminal and registration processing of registering time information indicating a time at which the notification request is made in a database as estrus history of the livestock.

In addition, according to the present disclosure, there is provided an information processing system including: a breeder-side information terminal configured to display a transition of a state quantity showing a characteristic change in an estrus period of livestock; an information processing device that includes a notification control unit configured to execute, in accordance with a notification request to cause estrus of the livestock to be notified to a veterinarian-side information terminal on a basis of an operation of the breeder-side information terminal, both notification processing to the veterinarian-side information terminal and registration processing of registering time information indicating a time at which the notification request is made in a database as estrus history of the livestock; and the veterinarian-side information terminal configured to receive the notification and perform a notification operation.

Further, according to the present disclosure, there is provided an information processing method including: causing a transition of a state quantity showing a characteristic change in an estrus period of livestock to be displayed on a display unit of a breeder-side information terminal of a breeder of the livestock; and executing, in accordance with a notification request to cause estrus of the livestock to be notified to a veterinarian-side information terminal on a basis of an operation of the breeder-side information terminal, both notification processing to the veterinarian-side information terminal and registration processing of registering time information indicating a time at which the notification request is made in a database as estrus history of the livestock.

### Advantageous Effects of Invention

According to the present disclosure as described above, it is possible to determine the estrus of individual livestock while viewing a transition of the state quantity showing a characteristic change in the estrus period of livestock, and to execute both contact with a veterinarian and accumulation of information of estrus history by performing an operation of notifying the veterinarian of the estrus of the livestock.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is an explanatory diagram which describes an outline of an information processing system according to one embodiment of the present disclosure.

FIG. 2 is an explanatory diagram which shows a configuration of the information processing system according to the embodiment.

FIG. 3 is a block diagram which shows a functional configuration of the information processing system according to the embodiment.

FIG. 4 is an explanatory diagram in which an example of information included in a signal transmitted by the signal transmission device is shown.

FIG. 5 is an explanatory diagram in which an example of information included in a signal transmitted by a signal relay device is shown.

FIG. 6 is an explanatory diagram which shows an example of information included in a signal transmitted to a network connection device.

FIG. 7 is a block diagram which shows a mechanism configuration of an information processing server according to the embodiment.

FIG. 8 is a chart which shows basic information among individual information of livestock.

FIG. 9 is a chart which shows breeding information among the individual information of livestock.

FIG. 10 is an explanatory diagram which shows a breeding cycle of dairy cattle.

FIG. 11 is an explanatory diagram which shows a relationship between changes in metabolic rate and states of livestock (dairy cattle).

FIG. 12 is a block diagram which shows an example of a hardware configuration of an information processing device of the information processing system according to the present embodiment.

FIG. 13 is an explanatory diagram which shows a processing operation performed in the information processing system according to the present embodiment.

FIG. 14 is an explanatory diagram which shows an example of an initial screen displayed on a breeder-side information terminal.

FIG. 15 is an explanatory diagram which shows an example of management information displayed on the breeder-side information terminal.

FIG. 16 is an explanatory diagram which shows an example of breeding information displayed on the breeder-side information terminal.

FIG. 17 is an explanatory diagram which shows a display example of an activity amount.

FIG. 18 is an explanatory diagram which shows a display example of an activity amount including a display of a predicted estrus data.

FIG. 19 is an explanatory diagram which shows a display example of an activity amount including a display of a predicted transition of an activity amount.

FIG. 20 is an explanatory diagram which shows a display example of an activity amount including a display of a reference transition.

FIG. 21 is an explanatory diagram which shows an example of management information displayed on the breeder-side information terminal after an estrus contact with a veterinarian.

FIG. 22 is an explanatory diagram which shows an example of management information displayed on the breeder-side information terminal after the estrus contact with a veterinarian.

FIG. 23 is an explanatory diagram which shows an example of a notification display which is displayed on a veterinarian-side information terminal.

FIG. 24 is an explanatory diagram which shows an example of management information displayed on the veterinarian-side information terminal.

FIG. 25 is an explanatory diagram which shows an example of breeding information displayed on the veterinarian-side information terminal after an estrus contact is received.

FIG. 26 is an explanatory diagram which shows an example of breeding information displayed on the veterinarian-side information terminal.

FIG. 27 is an explanatory diagram which shows a processing operation performed in the information processing system according to the embodiment.

FIG. 28 is an explanatory diagram which shows an example of breeding information displayed on the breeder-side information terminal after estrus diagnosis.

FIG. 29 is an explanatory diagram which shows an example of breeding information displayed on the veterinarian-side information terminal after estrus diagnosis.

FIG. 30 is an explanatory diagram which shows an example of breeding information displayed on the breeder-side information terminal after pregnancy diagnosis.

FIG. 31 is an explanatory diagram which shows an example of breeding information displayed on the veterinarian-side information terminal after pregnancy diagnosis.

FIG. 32 is an explanatory diagram which shows an example of breeding information displayed on the veterinarian-side information terminal after sex identification.

FIG. 33 is an explanatory diagram which shows an example of breeding information displayed on the breeder-side information terminal after sex identification.

FIG. 34 is an explanatory diagram which shows display examples of medication history information and injury/disease history information.

FIG. 35 is an explanatory diagram which shows a display example of milk component analysis information.

FIG. 36 is a flowchart which shows an example of state determination processing executed by the information processing device according to the embodiment.

### MODE(S) FOR CARRYING OUT THE INVENTION

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

Note that the description will be made in the following order.

1. Information processing system
1-1. Outline of information processing system
1-2. Configuration of information processing system
2. Information processing device
2-1 Communication network connection unit
2-2 Database storage unit
2-3 Control unit
2-4 Hardware configuration of information processing device
3. Operation of information processing system
4. Positive list information
5. State determination method

### 6. Summary

### 1. INFORMATION PROCESSING SYSTEM

### <1-1. Outline of Information Processing System>

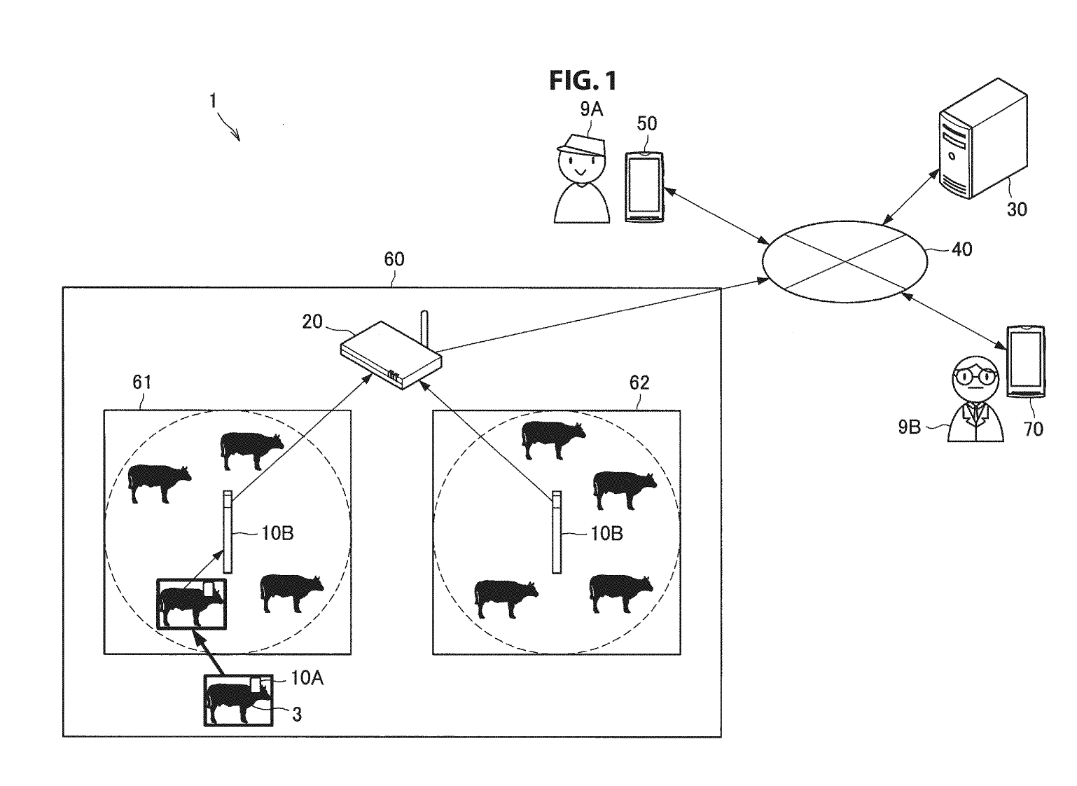

First, an outline of an information processing system 1 according to one embodiment of the present disclosure will be described with reference to FIG. 1. FIG. 1 is an explanatory diagram which shows the outline of the information processing system 1 according to the present embodiment.

As shown in FIG. 1, in the information processing system 1, a signal transmitted by a signal transmission device 10A attached to livestock 3 is received by a signal relay device 10B installed to correspond to an individual breeding area (for example, a barn 61, 62, or the like) which corresponds to a breeding stage of a pasture 60, and is transmitted to an information processing server 30 via a communication network connection device 20. The information processing server 30 can be accessed by a breeder-side information terminal 50 used by a breeder (for example, a dairy farmer) 9A, and a veterinarian-side information terminal 70 used by a veterinarian 9B via a communication network 40.

The livestock 3 may be, for example, an industrial animal such as a beef cow, a dairy cow, a pig, a horse, a sheep, a goat, or a poultry bird, or may be a companion animal such as a dog, a cat or a rabbit. The livestock 3 is bred in a breeding area such as a barn 61 or 62 of a pasture 60. In addition, different types of breeding and management are performed on livestock 3 in each breeding area on the basis of individual information such as an age in month, a sex, a breed, a health state and a breeding cycle. Note that a case in which the livestock 3 is a dairy cow will be described as an example in the present specification.

A signal which has reached the information processing server 30 includes a transmission device identifier for identifying the signal transmission device 10A which has transmitted a signal, and a relay device identifier for identifying the signal relay device 10B which has received a signal. Therefore, the information processing server 30 determines which signal relay device 10B a signal transmitted by the signal transmission device 10A has passed through to reach, thereby determining a breeding area in which the livestock 3 to which the signal transmission device 10A is attached is located.

For example, in a case in which the livestock 3 to which the signal transmission device 10A is attached enters the barn 61, the signal transmission device 10A transmits a signal including a transmission device identifier for identifying itself and the transmitted signal is received by the signal relay device 10B having a communicable area inside the barn 61. The signal relay device 10B adds a relay device identifier for identifying itself to the received signal, and transmits the added signal to the information processing server 30. The information processing server 30 can detect a position of the livestock 3 on the basis of individual information of the livestock 3 corresponding to a transmission device identifier included in a received signal (that is, the livestock 3 to which the signal transmission device 10A is attached) and a relay device identifier.

Moreover, the signal transmitted by the signal transmission device 10A includes one or more of various pieces of sensor information detected by one or a plurality of sensors included in the signal transmission device 10A. The sensor information includes at least information correlated with a predetermined state quantity showing a characteristic change in the estrus period. For example, since the livestock 3 has an increased activity amount in the estrus period, vibration information including the magnitude and frequency of vibration correlated with the activity amount of the livestock 3 may be included as sensor information. The information processing system 1 according to the present embodiment may further include temperature information including a body temperature correlated with the metabolic rate of the livestock 3 as sensor information.

In addition to this, the information processing server 30 may store basic information and breeding information of the livestock 3 which are input by the breeder 9A and the veterinarian 9B as individual information of the livestock 3. In a case in which predetermined display information is requested by the breeder-side information terminal 50 or the veterinarian-side information terminal 70, the information processing server 30 transmits display information corresponding to the request to the breeder-side information terminal 50 or the veterinarian-side information terminal 70 which has made the request. That is, information of each head of the livestock 3 can be shared by the breeder 9A and the veterinarian 9B. The display information is information for performing a screen display, and includes text information, numerical information, image information, and the like.

The information processing server 30 can transmit at least breeding information including a display of a transition of the state quantity showing a characteristic change in the estrus period to the breeder-side information terminal 50 or the veterinarian-side information terminal 70 which has made the request. In the information processing system 1 according to the present embodiment, the activity amount of the livestock 3 is used as the state quantity. Therefore, the breeder 9A can determine estrus of the livestock 3 while viewing a transition of the activity amount of individual livestock 3. In addition, the breeder 9A performs an operation of estrus contact to inform the veterinarian 9B of the estrus of the livestock 3, and thereby the information processing server 30 causes information of an estrus date to be newly registered and accumulated while notifying the veterinarian-side information terminal 70 of the estrus of the livestock 3. As a result, when livestock 3 is in the estrus state, the breeder 9A can promptly notify the veterinarian 9B of the estrus of the livestock 3, and information of an actual estrus date can be accumulated in the information processing server 30.

Note that, in the following description, information for identifying the signal transmission device 10A is referred to as a transmission device identifier, and a signal transmitted from the signal transmission device 10A is referred to as a transmission signal in the following description. In addition, information for identifying the signal relay device 10B is referred to as a relay device identifier, and a signal transmitted from the signal relay device 10B is referred to as a relay signal.

### <1-2. Configuration of Information Processing System>

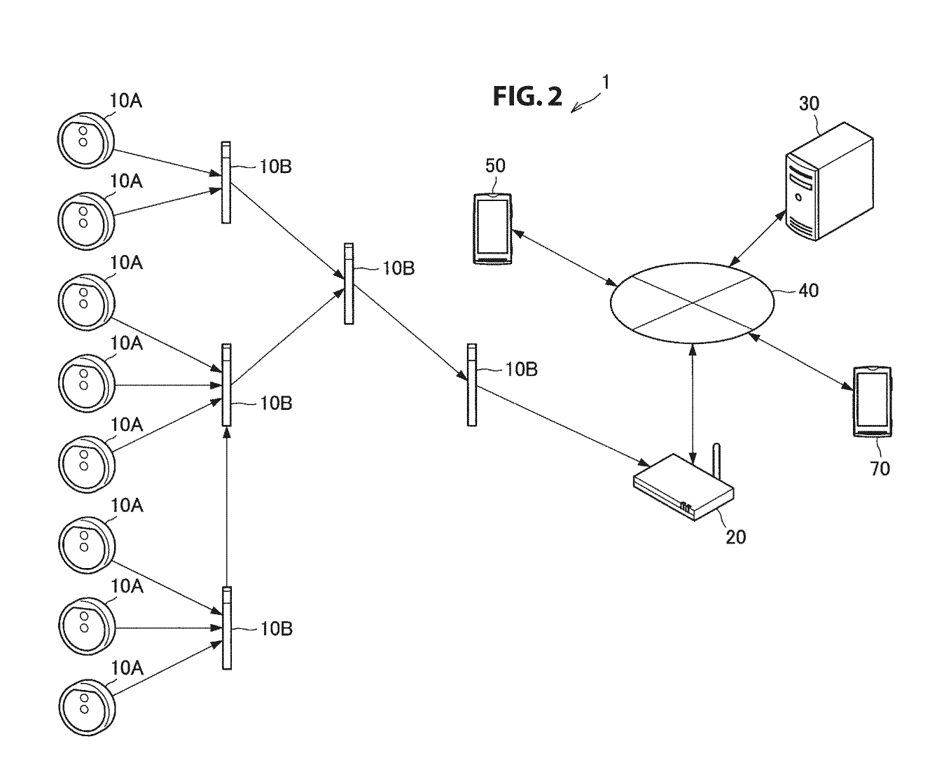

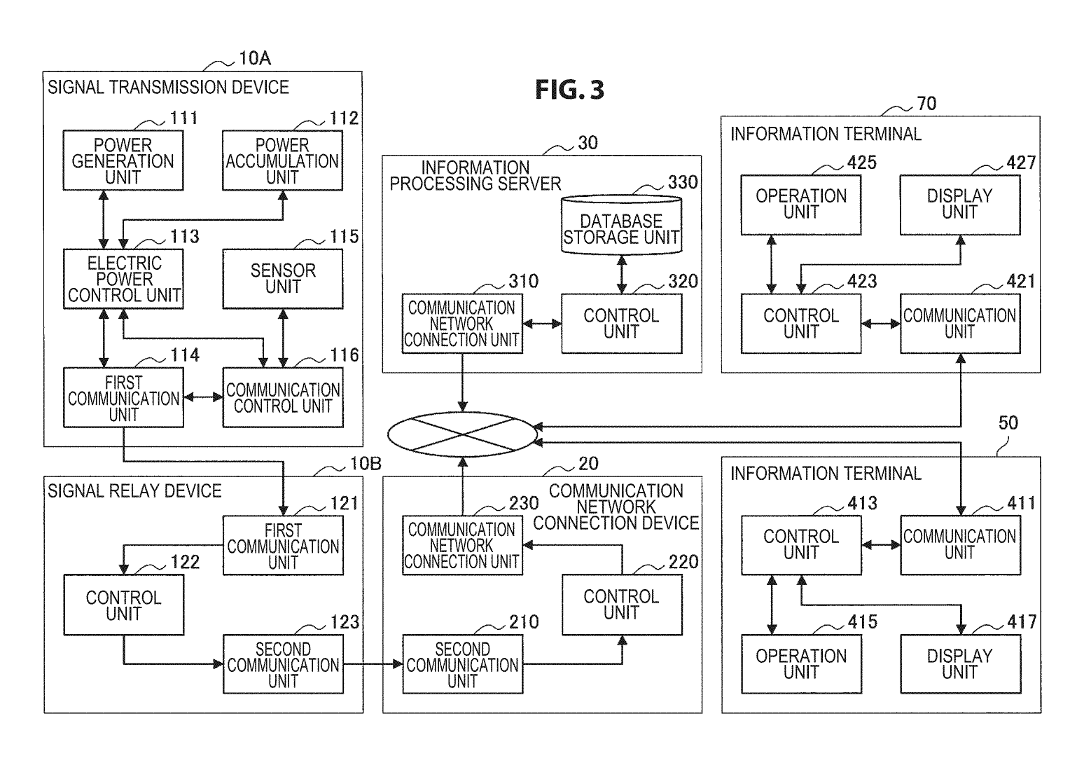

Next, a configuration example of the information processing system 1 according to the present embodiment will be described with reference to FIGS. 2 and 3. FIG. 2 is an explanatory diagram which shows a configuration example of the information processing system 1 according to the present embodiment. FIG. 3 is a block diagram which shows a functional configuration of the information processing system 1 according to the present embodiment.

The information processing system 1 includes a signal transmission device 10A, a signal relay device 10B, a communication network connection device 20, an information processing server 30, a breeder-side information terminal 50, and a veterinarian-side information terminal 70. The communication network connection device 20, the information processing server 30, the breeder-side information terminal 50, and the veterinarian-side information terminal 70 can communicate with each other via a communication network 40.

### [1-2-1. Signal Transmission Device]

The signal transmission device 10A is attached to each head of the livestock 3 and transmits a transmission signal at a predetermined timing (for example, at a cycle of tens of seconds). A transmission signal transmitted by the signal transmission device 10A includes a transmission device identifier for identifying the signal transmission device 10A, and the information processing system 1 can identify the signal transmission device 10A and the livestock 3 to which the signal transmission device 10A is attached according to the transmission device identifier. For example, the signal transmission device 10A may be an identification tag attached to the ear and the like of the livestock 3. Note that the signal transmission device 10A may be directly attached to the livestock 3 by affixing it to or embedding it in the body of the livestock 3, or may be indirectly attached to the livestock 3 using an attachment case or the like.

As shown in FIG. 3, the signal transmission device 10A includes a power generation unit 111, a power accumulation unit 112, an electric power control unit 113, a first communication unit 114, a sensor unit 115, and a communication control unit 116.

### (1-2-1-1. Power Generation Unit)

The power generation unit 111 includes one or a plurality of power generation devices capable of generating electric power in accordance with a surrounding environment of the signal transmission device 10A. Specifically, the power generation unit 111 may be one or more of various power generation devices such as a vibration power generation device, a photovoltaic device, a thermoelectric conversion power generation device, an enzyme power generation device, a radio wave power generation device, and a near electromagnetic power generation device. The signal transmission device 10A includes the power generation unit 111, and thereby transmits a transmission signal using electric power generated by the power generation unit 111 even without including a power source such as a battery. As a result, since the signal transmission device 10A can transmit a transmission signal even without including a battery and the like, portability can be improved and a load on the livestock 3 can be reduced. In addition, since a battery is not included, it is possible to save trouble of managing a battery.

The vibration power generation device includes a power generation element of an electrostatic type, an electromagnetic type, a reverse magnetostrictive type, or a piezoelectric type, and is a power generation device that generates power using vibration. The photovoltaic device is, for example, a power generation device that generates power using sunlight or indoor light. For example, the thermoelectric conversion power generation device includes a power generation element using a Seebeck effect or a Thomson effect, a thermos-electronic power generation element, or a thermomagnetic power generation element, and is a power generation device that generates power using heat, a temperature difference, or the like. An enzyme power generation device is a power generation device that generates power by decomposing carbohydrates (for example, glucose and the like) contained in an organic matter and the like using enzymes. The radio wave power generation device is a power generation device that generates power using a radio wave such as Wi-Fi or terrestrial digital wave. The near electromagnetic field power generation device is a power generation device that generates power using, for example, an electromagnetic wave in a near field.

### (1-2-1-2. Power Accumulation Unit)

The power accumulation unit 112 accumulates power generated by the power generation unit 111. The power accumulated in the power accumulation unit 112 is used to cause the first communication unit 114 to operate, for example. The power accumulation unit 112 may be one of various secondary batteries such as a lithium ion secondary battery, various capacitors such as an electric double layer capacitor or a lithium ion capacitor, and various capacitors such as a ceramic capacitor, a film capacitor, an aluminum electrolytic capacitor, and a tantalum capacitor, or a combination of a plurality of them.

### (1-2-1-3. Electric Power Control Unit)

The electric power control unit 113 controls electric power supply inside the signal transmission device 10A. The electric power control unit 113 can be constituted by an arithmetic operation processing device such as a micro processing unit (MPU) or a central processing unit (CPU), and memories such as a read only memory (ROM) and a random access memory (RAM). Specifically, the electric power control unit 113 charges the power accumulation unit 112 with electric power generated by the power generation unit 111, and, in a case in which the electric power stored in the power accumulation unit 112 reaches a predetermined amount, supplies electric power to the first communication unit 114 from the power accumulation unit 112. As a result, the signal transmission device 10A can transmit a transmission signal from the first communication unit 114 every time an amount of electric power generated by the power generation unit 111 reaches a predetermined amount. A timing at which a transmission signal is transmitted from the signal transmission device 10A depends on a power generation amount per unit time in the power generation unit 111, but may be, for example, at a cycle of tens of seconds.

### (1-2-1-4. Sensor Unit)

The sensor unit 115 detects a state of the livestock 3 or a state of the surroundings of the livestock 3. The sensor unit 115 may include a vibration sensor, a first temperature sensor, and a second temperature sensor. Among these sensors, the vibration sensor detects vibration occurring according to an operation of the livestock 3. An amount of detected vibration is correlated with the activity amount of the livestock 3, and thus is used to calculate the activity amount. For example, the vibration sensor is constituted using a piezoelectric element, and generates electric power in accordance with a magnitude and frequency of vibration. The vibration sensor may also function as the power generation unit 111. At least some of electric power generated by the vibration sensor may be accumulated in a power accumulation device for vibration power generation as information (vibration information) indicating a presence or absence and magnitude of vibration. The power accumulation device may also be a power accumulation device with a smaller capacity than a power accumulation device for accumulating electric power for communication.

Electric power accumulated in the power accumulation device for vibration power generation, that is, an inter-terminal voltage of the power accumulation device for vibration power generation shows different values in accordance with the frequency and magnitude of vibration per unit time detected by the vibration sensor. Greater vibration or a greater frequency of vibration detected by the vibration sensor results in a greater value of the inter-terminal voltage. Information of electric power generated and accumulated by the vibration sensor is included in a transmission signal of the signal transmission device 10A as vibration information and transmitted to the information processing server 30. The information processing server 30 can calculate information of the activity amount of the livestock 3 on the basis of the vibration information.

In addition, the first temperature sensor is provided, for example, in proximity to a body surface of the livestock 3, and detects a first temperature T1 that can vary with a body temperature of the livestock 3. The second temperature sensor is provided, for example, in a direction opposite to the body surface of the livestock 3, and detects a second temperature T2 that can vary with the outside air temperature. The detected temperature information is included in the transmission signal of the signal transmission device 10A, and transmitted to the information processing server 30. The information processing server 30 can detect information of the metabolic rate of the livestock 3 on the basis of a temperature difference ΔT between the first temperature T1 and the second temperature T2. In this case, a greater value of the temperature difference ΔT shows a high metabolic rate of the livestock 3.

The sensor unit 115 may further include sensors other than the vibration sensor, the first temperature sensor, and the second temperature sensor described above, or in conjunction with the vibration sensor, the first temperature sensor, and the second temperature sensor. For example, the sensor unit 115 may include a sensor that measures the state of the livestock 3 such as a body temperature sensor, a heart rate sensor, a blood glucose sensor, and a blood pressure sensor. In addition, the sensor unit 115 may include a sensor that measures the state of a surrounding environment of livestock such as a temperature sensor, a humidity sensor, an atmospheric pressure sensor, an illumination sensor, vibration sensors such as an acceleration sensor and a gyro sensor, a geomagnetic sensor, a microphone, and an imaging device. Moreover, the signal transmission device 10A may include a plurality of types of the sensors described above. As long as a sensor can detect information correlated with the state quantity showing a characteristic change in the estrus period of the livestock 3, there is no particular restriction on a sensor that can be used.

### (1-2-1-5. First Communication Unit)

The first communication unit 114 includes an antenna and a communication circuit, and transmits a transmission signal to the signal relay device 10B. Specifically, the first communication unit 114 transmits a transmission signal including a transmission device identifier for identifying the signal transmission device 10A using electric power accumulated in the power accumulation unit 112 in a case in which the electric power accumulated in the power accumulation unit 112 reaches a predetermined amount. The first communication unit 114 may also include an antenna and a communication circuit that perform communication using signals of wavelengths in a band of hundreds of MHz to several GHz (for example, 920 MHz and the like) such as Wi-Fi (registered trademark), ZigBee (registered trademark), Bluetooth (registered trademark), Bluetooth Low Energy (registered trademark), ANT (registered trademark), ANT+(registered trademark), and EnOcean Alliance (registered trademark), or mobile communication such as 3G or Long Term Evolution (LTE). Alternatively, the first communication unit 114 may also include an antenna and a communication circuit corresponding to Bluetooth Low Energy.

### (1-2-1-6. Communication Control Unit)

The communication control unit 116 controls transmission of a signal by the first communication unit 114. The communication control unit 116 can be constituted by, for example, an arithmetic operation processing device such as an MPU or a CPU, and memories such as a ROM and a RAM.

Specifically, the communication control unit 116 controls the first communication unit 114 such that it transmits a transmission signal including a transmission device identifier and sensor information using electric power accumulated in the power accumulation unit 112. That is, the communication control unit 116 controls the first communication unit 114 such that it causes various pieces of sensor information detected by the sensor unit 115 to be added to a transmission signal and then transmits the transmission signal in a case in which electric power accumulated in the power accumulation unit 112 has reached a predetermined amount.

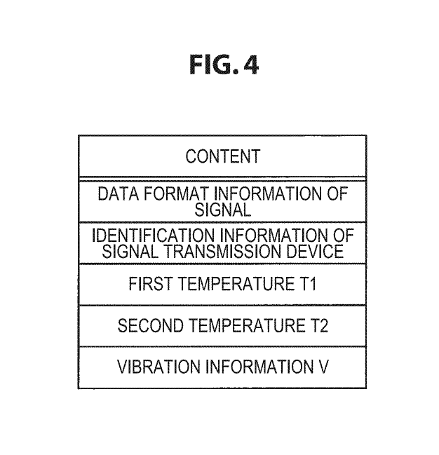

FIG. 4 is an explanatory diagram which shows an example of information included in a transmission signal of the signal transmission device 10A. As shown in FIG. 4, the transmission signal of the signal transmission device 10A may include, for example, data format information of a signal indicating what type of information is included in the relay signal, a transmission device identifier for identifying the signal transmission device 10A which has transmitted a transmission signal, and various pieces of sensor information measured by the sensor unit 115. The various pieces of sensor information may include information of a first temperature T1 detected by the first temperature sensor, information of a second temperature T2 detected by the second temperature sensor, and vibration information detected by the vibration sensor

### [1-2-2. Signal Relay Device]

The signal relay device 10B is installed in a predetermined area of the pasture 60 (for example, a barn 61, 62, or the like), and receives a transmission signal from the signal transmission device 10A. In addition, the signal relay device 10B transmits a relay signal obtained by adding a relay device identifier for identifying the signal relay device 10B to the received transmission signal to the communication network connection device 20. At this time, the relay signal may be directly transmitted to the communication network connection device 20, or may also be transmitted to the communication network connection device 20 via at least one other signal relay device 10B.

For example, the signal relay device 10B may be a dedicated communication device having a cylindrical shape as shown in FIG. 2, may be a communication device having the same configuration as the signal transmission device 10A, or may be a general portable communication terminal such as a mobile phone or a smart phone.

As shown in FIG. 3, the signal relay device 10B includes a first communication unit 121, a control unit 122, and a second communication unit 123.

### (1-2-2-1. First Communication Unit)

The first communication unit 121 includes an antenna and a communication circuit, and receives a transmission signal from the signal transmission device 10A. For example, the first communication unit 121 may be constituted by an antenna and a communication circuit which can receive transmission signals from the first communication unit 114 of the signal transmission device 10A.

### (1-2-2-2. Second Communication Unit)

The second communication unit 123 includes an antenna and a communication circuit, and transmits relay signals to the communication network connection device 20. Specifically, the second communication unit 123 transmits a relay signal obtained by adding a relay device identifier for identifying the signal relay device 10B to a transmission signal received by the first communication unit 121 to the communication network connection device 20. As a result, the information processing server 30 which has received the relay signal can identify the signal transmission device 10A which has transmitted a transmission signal and the signal relay device 10B which has received a transmission signal according to a transmission device identifier and a relay device identifier included in the relay signal.

The second communication unit 123 may include an antenna and a communication circuit that perform communication using signals of wavelengths in a band of hundreds of MHz to several GHz (for example, 920 MHz and the like) such as Wi-Fi, ZigBee, Bluetooth, Bluetooth Low Energy, ANT, ANT+, and EnOcean Alliance, or mobile communication such as 3G or LTE. In addition, the second communication unit 123 may also include an antenna and a communication circuit which are different from those of the first communication unit 121 in at least one of the frequency or communication method. According to this, the signal relay device 10B can prevent a transmission signal and a relay signal from being confused, and a communication unit of a relay signal transmitted from the signal relay device 10B may be a wireless communication unit or a wired communication unit.

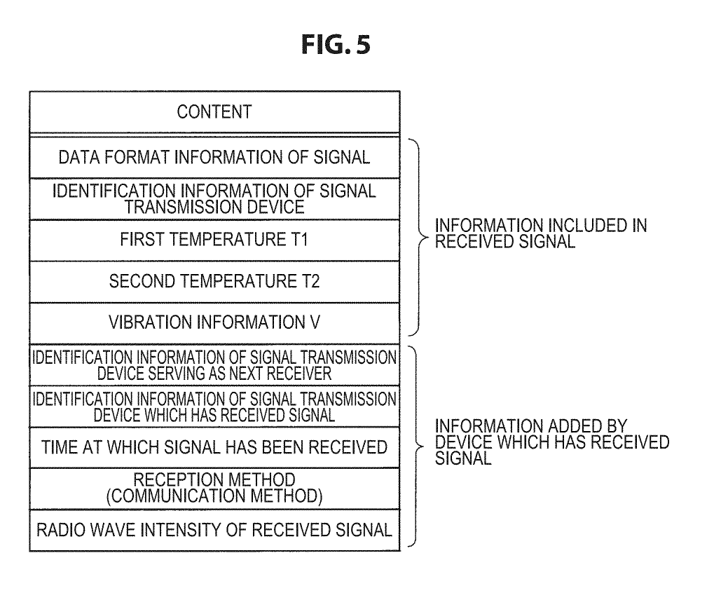

FIG. 5 is an explanatory diagram which shows an example of information included in a relay signal transmitted by the signal relay device 10B. As shown in FIG. 5, the relay signal transmitted by the signal relay device 10B may include, for example, in the same manner as in FIG. 4, data format information of a signal indicating what type of information is included in the relay signal, a transmission device identifier for identifying the signal transmission device 10A which has transmitted a transmission signal, and various pieces of sensor information measured by the sensor unit 115. The various pieces of sensor information may include information of a first temperature T1 detected by the first temperature sensor, information of a second temperature T2 detected by the second temperature sensor, and vibration information detected by the vibration sensor. In addition, the relay signal transmitted by the signal relay device 10B may include, for example, an identifier for identifying a signal relay device which is a next receiver, a relay device identifier for identifying the signal relay device 10B which has received the transmission signal, information indicating a time at which the transmission signal has received, and information indicating a radio wave intensity of a received signal. These pieces of information are information added to the relay signal by the signal relay device 10B.

### (1-2-2-3. Control Unit)

The control unit 122 controls each unit of the signal relay device 10B. Specifically, the control unit 122 adds a relay device identifier for identifying the signal relay device 10B to the transmission signal received by the first communication unit 121, and controls each unit such that the second communication unit 123 transmits the transmission signal with the relay device identifier as a relay signal. The control unit 122 may be constituted by, for example, an arithmetic processing device such as an MPU or a CPU, and memories such as a ROM and a RAM that store identification information of the signal relay device 10B, a control program, control parameters, and the like.

In addition, the control unit 122 may add information of the radio wave intensity of a signal when a transmission signal transmitted by the signal transmission device 10A is received by the first communication unit 121 to the transmission signal, and sets the transmission signal with the information as a relay signal. Accordingly, since the information processing server 30 can estimate a distance between the signal transmission device 10A and the signal relay device 10B on the basis of the radio wave intensity of a transmission signal, it is possible to ascertain the position of the signal transmission device 10A in more detail. In addition, the information processing server 30 can remove a noise signal and determine the position of the signal transmission device 10A more accurately by ignoring a transmission signal whose radio wave intensity is less than a threshold value.

### (1-2-3. Communication Network Connection Device)

The communication network connection device 20 transmits a relay signal transmitted from each signal relay device 10B to the information processing server 30 via the communication network 40. The communication network connection device 20 may be a communication device including any one of wired and wireless communication devices as long as the communication network connection device 20 can be connected to the communication network 40. For example, the communication network connection device 20 may be a gateway device which can be connected to a wired or wireless local area network (LAN), or a mobile communication terminal which can be connected to a mobile communication network.

As shown in FIG. 3, the communication network connection device 20 includes a second communication unit 210, a control unit 220, and a communication network connection unit 230.

### (1-2-3-1. Second Communication Unit)

The second communication unit 210 includes an antenna and a communication circuit, and receives relay signals transmitted from the signal relay device 10B. Specifically, the second communication unit 210 may be constituted by an antenna and a communication circuit capable of receiving relay signals transmitted from the second communication unit 123 of the signal relay device 10B.

### (1-2-3-2. Control Unit)

The control unit 220 controls each unit of the communication network connection device 20. Specifically, the control unit 220 controls the transmission and reception of a signal in the second communication unit 210 and the communication network connection unit 230. The control unit 220 may include, for example, an MPU that is an arithmetic operation processing device, and a memory that stores a control program, control parameters, and the like.

### (1-2-3-3. Communication Network Connection Unit)

The communication network connection unit 230 transmits a relay signal received by the second communication unit 210 to the information processing server 30. If the communication network connection unit 230 may be either a wired or wireless communication device as long as it can be connected to the communication network 40. For example, the communication network connection unit 230 may be a wired or wireless LAN compatible communication device, may be a cable communication device that performs wired cable communication, or may be a communication device that performs mobile communication.

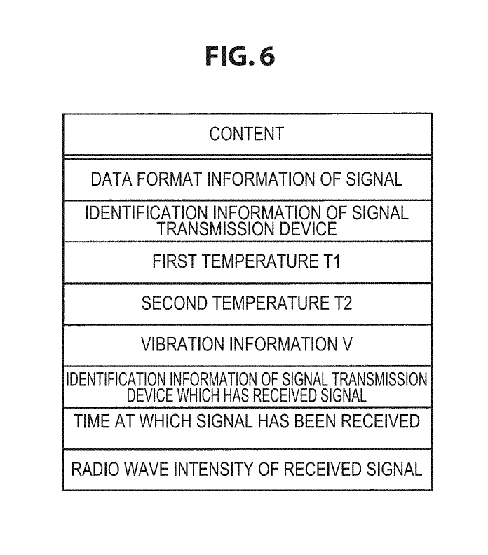

FIG. 6 shows an example of information included in a relay signal transmitted by the communication network connection device 20. As shown in FIG. 6, the relay signal transmitted by the communication network connection device 20 can include, for example, in the same manner as in FIG. 5, data format information of a signal indicating what type of information is included in the relay signal, a transmission device identifier for identifying the signal transmission device 10A that has transmitted a transmission signal, and various pieces of sensor information detected by the sensor unit 115. The sensor information can include at least information of a first temperature T1, information of a second temperature T2, and vibration information.

Moreover, the relay signal transmitted by the communication network connection device 20 may include, for example, a relay device identifier for identifying the signal relay device 10B that is passed through, information indicating a time at which the signal relay device 10B has received a transmission signal, and information indicating a radio wave intensity of the received transmission signal. Note that identification information for identifying a signal relay device serving as a next receiver may be included in a signal transmitted by the communication network connection device 20.

### [1-2-4. Communication Network]

The communication network 40 is a network in which information is transmitted or received. The communication network 40 may be, for example, the Internet, a satellite communication network, a telephone line network, a mobile communication network (for example, a 3G network and the like), or the like.

### [1-2-5. Breeder-Side Information Terminal]

The breeder-side information terminal 50 is used by a breeder, and is capable of accessing the information processing server 30 via the communication network 40. The breeder-side information terminal 50 receives an operation input of a user (breeder), and displays display image including various pieces of reference information for managing a cattle group on a display unit 417. As shown in FIG. 3, the breeder-side information terminal 50 includes a communication unit 411, a control unit 413, an operation unit 415, and the display unit 417. The breeder-side information terminal 50 may include an audio output device such as a speaker and a headphone.

The operation unit 415 includes an input device to which a user (breeder) inputs operation command information, such as a touch panel, a keyboard, a mouse, a button, a microphone, a switch, and a lever, and an input control circuit for generating an input signal on the basis of input information. The operation unit 415 converts an input from a user (breeder) into an input signal and transmits it to a control unit 520.

The display unit 417 may include, for example, a display device such as a cathode ray tube (CRT) display device, a liquid crystal display device, and an organic electro-luminescence (EL) display device. For example, the display unit 417 displays a display image generated on the basis of display information received from the information processing server 30.

The communication unit 411 includes an antenna and a communication circuit for connecting to the communication network 40, receives display information from the information processing server 30, and transmits input information to the operation unit 415 by a user to the information processing server 30. For example, the communication unit 411 may be a wired or wireless LAN compatible communication device, may be a cable communication device that performs wired cable communication, or may also be a communication device that performs mobile communication.

The control unit 413 controls each unit of the breeder-side information terminal 50. For example, the control unit 413 causes the information processing server 30 to output a signal requesting display information via the communication unit 411 in accordance with an input operation to the operation unit 415. In addition, the control unit 413 causes the information processing server 30 to output a notification command signal to the veterinarian-side information terminal 70 via the communication unit 411 in accordance with an input operation to the operation unit 415. Moreover, the control unit 413 causes the display unit 417 to display the display information transmitted from the information processing server 30 via the communication network 40. The control unit 413 may include an arithmetic operation processing device such as an MPU or a CPU, and memories such as a ROM and a RAM that store control programs, control parameters, and the like. In this case, an arithmetic processing device executes an application stored in a storage medium such as a memory, and thereby each operation of the control unit 413 may be executed.

### [1-2-6. Veterinarian-Side Information Terminal]

The veterinarian-side information terminal 70 is used by a veterinarian, and can access the information processing server 30 via the communication network 40. The veterinarian-side information terminal 70 receives an operation input by a user (veterinarian), and displays a display image including various pieces of reference information for managing a cattle group on a display unit 427. As shown in FIG. 3, the veterinarian-side information terminal 70 includes a communication unit 421, a control unit 423, an operation unit 425, and the display unit 427.

Each of the communication unit 421, the control unit 423, the operation unit 425, and the display unit 427 can be constituents the same as those of each unit of the breeder-side information terminal 50. However, even if common information used for management of the livestock 3 is requested to the information processing server 30 from the breeder-side information terminal 50 and the veterinarian-side information terminal 70, since roles of a breeder and a veterinarian are different, display content on the display unit 417 of the breeder-side information terminal 50 can be different from display content on the display unit 427 of the veterinarian-side information terminal 70. In the veterinarian-side information terminal 70, each operation of the control unit 423 may also be executed by an arithmetic processing device executing an application recorded in a storage medium such as a memory.

### [1.2.7. Information Processing Server]

The information processing server 30 receives a relay signal transmitted from the signal relay device 10B, and manages individual information of each head of the livestock 3. The relay signal transmitted to the information processing server 30 can include a transmission device identifier of a signal transmission device 10A that has transmitted a transmission signal, information indicating a time at which a signal relay device 10B has received the transmission signal, and sensor information such as vibration information or temperature information. The information processing server 30 accumulates received time information and sensor information together with a transmission device identifier. As the time information, a time at which the information processing server 30 has received a signal may be added instead of a time at which a signal relay device 10B has received a transmission signal. In addition, the information processing server 30 receives information input by a breeder or a veterinarian to the breeder-side information terminal 50 and the veterinarian-side information terminal 70 via the communication network 40, and manages individual information of each head of the livestock 3. For example, the information input to the breeder-side information terminal 50 and the veterinarian-side information terminal 70 may be at least one of information of a estrus period of each head of the livestock 3, information of a result of estrus diagnosis, information of a result of pregnancy diagnosis, information of a result of sex identification, and information of a result of childbirth.

The information processing server 30 causes the breeder-side information terminal 50 and the veterinarian-side information terminal 70 to display predetermined display information in accordance with requests from the breeder-side information terminal 50 and the veterinarian-side information terminal 70. As a result, a breeder and a veterinarian can share individual information of the livestock 3. In addition, the information processing server 30 can perform notification processing to the other information terminal in accordance with an input from one of the breeder-side information terminal 50 and the veterinarian-side information terminal 70.

For example, the information processing server 30 transmits display information of basic breeding information of specific livestock 3 to the breeder-side information terminal 50 and causes the display information to be displayed on the breeder-side information terminal 50 when a display request for the basic breeding information is received from the breeder-side information terminal 50. In addition, in a case in which a breeder determines estrus of livestock while referring to the basic breeding information, and inputs estrus contact, the information processing server 30 receives a notification request to the veterinarian-side information terminal 70, and performs notification processing to the veterinarian-side information terminal 70. At this time, the information processing server 30 registers time information indicating a time at which a notification request of the estrus contact is made in a database as an estrus history of the livestock. The time at which this notification request of the estrus contact is made may be a time at which a breeder has input the estrus contact to the breeder-side information terminal 50, or may be a time at which the information processing server 30 has received the notification request of the estrus contact. In addition, the time information may be information of estrus date, and may also include more detailed information such as the year, month, date, time, and second of estrus.

As shown in FIG. 3, the information processing server 30 includes a communication network connection unit 310, a control unit 320, and a database storage unit 330.

The communication network connection unit 310 includes a communication interface and a communication circuit which can be connected to the communication network 40, and receives a relay signal transmitted from a signal relay device 10B via the communication network 40. As long as the communication network connection unit 310 is a communication device which can be connected to the communication network 40 like the communication network connection unit 230 of the communication network connection device 20, and can transmit or receive signals via the communication network 40, either a wired or wireless communication method may be used. For example, the communication network connection unit 310 may be a wired or wireless LAN compatible communication device, may be cable communication device that performs wired cable communication, or may also be a communication device that performs mobile communication.

The control unit 320 includes an arithmetic processing device such as an MPU or a CPU, and memories such as a ROM and a RAM. The control unit 320 executes storage processing of sensor information in the database storage unit 330 on the basis of a relay signal generated by including a transmission signal transmitted from a signal transmission device 10A attached to individual livestock 3 and individual information of the livestock 3. In addition, the control unit 320 executes processing of registering individual information of the livestock 3 input to the breeder-side information terminal 50 and the veterinarian-side information terminal 70 in the database storage unit 330.

The database storage unit 330 is a storage unit that stores database in which the sensor information or the individual information of the livestock 3 is registered. The individual information of the livestock 3 may be, for example, the name, individual number, age in month, sex, breed, breeding cycle, and estrus history of the livestock 3, a transmission device identifier of an attached signal transmission device 10A, and the like. The estrus history includes information of an estrus date, an estrus time (hour and minute), an estrus duration time, or an estrus intensity. The sensor information is, for example, vibration information and temperature information which are detected by a temperature sensor and a vibration sensor included in a signal transmission device 10A and transmitted together with a transmission signal. For example, the database storage unit 330 may be a storage device such as a hard disk drive (HDD) device or a solid state drive (SSD) device. Note that the database storage unit 330 may be a separate storage server from the information processing server 30.

### 2. INFORMATION PROCESSING DEVICE (INFORMATION PROCESSING SERVER)

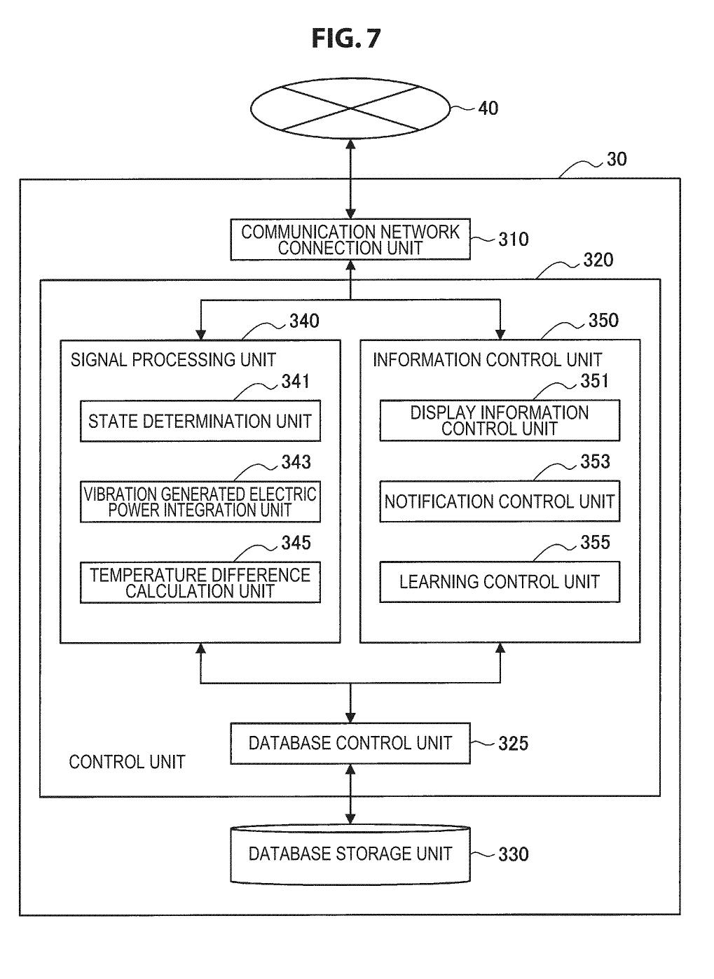

Next, the information processing server (information processing device) 30 according to the present embodiment will be described in detail with reference to FIG. 7. FIG. 7 is a block diagram which shows a functional configuration of the information processing server 30 according to the present embodiment. The information processing device 30 may be, for example, an information processing server, but the present embodiment is not limited to this example. The information processing device 30 may be an information terminal such as a tablet terminal.

As shown in FIG. 7, the information processing server 30 includes a communication network connection unit 310, a control unit 320, and a database storage unit 330. Among them, the control unit 320 includes a signal processing unit 340, an information control unit 350, and the database storage unit 330. In addition, the signal processing unit 340 includes a state determination unit 341, a vibration generated electric power integration unit 343, and a temperature difference calculation unit 345. In addition, the information control unit 350 includes a display information control unit 351, a notification control unit 353, and a learning control unit 355.

### <2-1. Communication Network Connection Unit>

The communication network connection unit 310 includes a communication circuit and communication interface which can be connected to the communication network 40, and receives a relay signal transmitted from a signal relay device 10B via the communication network 40. In addition, the communication network connection unit 310 receives signals transmitted from the breeder-side information terminal 50 and the veterinarian-side information terminal 70 via the communication network 40. In addition, the communication network connection unit 310 transmits notification or display information to the breeder-side information terminal 50 or the veterinarian-side information terminal 70 via the communication network 40 on the basis of a command signal from the breeder-side information terminal 50 or the veterinarian-side information terminal 70.

### <2-2. Database Storage Unit>

The database storage unit 330 stores a database in which individual information for individual livestock 3 is registered. As described above, the database storage unit 330 may be a storage device such as an HDD device or an SSD device. Note that the database storage unit 330 may be a separate storage server from the information processing server 30.

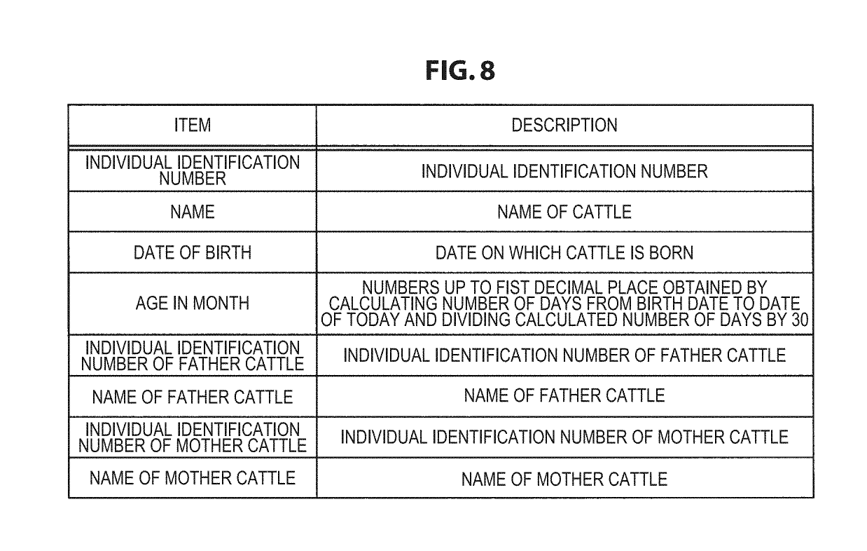

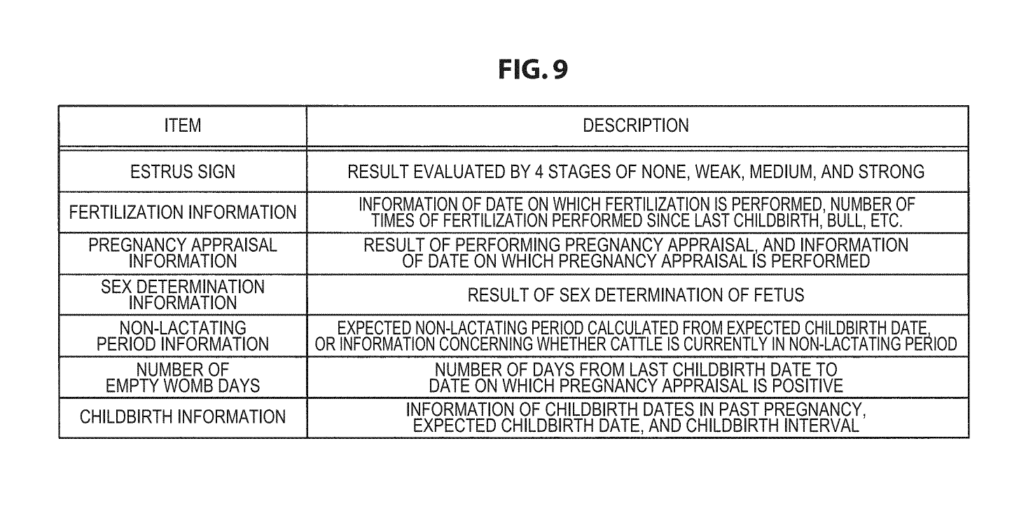

A specific example of individual information of the livestock 3 stored in the database storage unit 330 will be shown in FIGS. 8 and 9. FIG. 8 is a chart which shows basic information among individual information of dairy cattle serving as livestock 3, and FIG. 9 is a chart which shows breeding information of breeding among the individual information of dairy cattle serving as livestock 3. As shown in FIG. 8, the basic information of the livestock 3 stored in the database storage unit 330 can include, for example, an individual identification number of the livestock 3, a registration number corresponding to a name, an age in month, an individual identification number and a registration name of the father cattle, an individual identification number and a registration name of the mother cattle, and the like. In addition, as shown in FIG. 9, the breeding information of the livestock 3 stored in the database storage unit 330 can include a an estrus sign, fertilization information, pregnancy diagnosis information, sex identification information, non-lactating period information, the number of empty womb days, childbirth information, and the like of the livestock 3 as the breeding information of the livestock 3 stored in the database storage unit 330.

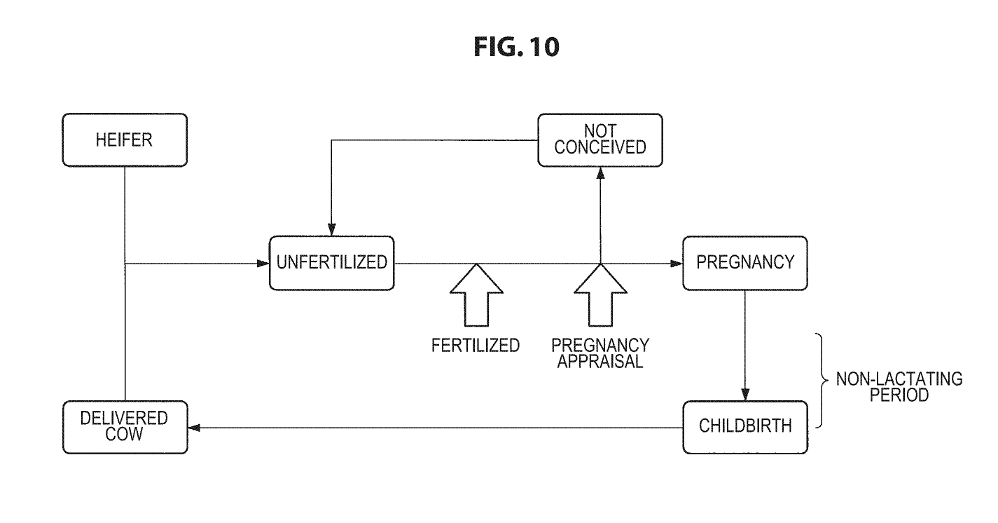

The details of these pieces of breeding information will be described with reference to FIG. 10. FIG. 10 is an explanatory diagram which shows a breeding cycle of dairy cattle. Note that the breeding cycle of dairy cattle is shown as an example of a breeding process of the livestock 3 in FIG. 10, but if livestock 3 is managed separately in breeding stages such as childhood, maturity, and a disease state, information of a stage of a breeding process corresponding to the livestock is stored in the database storage unit 330, and is used in each processing of the information processing server 30.

As shown in FIG. 10, in a case of dairy cattle, cattle with no childbirth experience is called a heifer, and cattle with childbirth experience is called a delivered cow. Both types of cattle become a pregnant state through occurrence of an estrus sign, fertilization, and pregnancy diagnosis first starting from an unfertilized state. Note that, in a case in which it is diagnosed that cattle is not conceived by pregnancy diagnosis, fertilization and pregnancy diagnosis are performed on the cattle again. In addition, milking is restricted in preparation for childbirth for 60 days before an expected childbirth date for cattle in a pregnant state. A period for which this milking is restricted is also called a non-lactating period. Cattle that gave birth after the non-lactating period returns to an unfertilized state again as a delivered cow. In dairy cattle, breeding is performed in the cycle as described above.

That is, the breeding information shown in FIG. 9 indicates in which state of the breeding cycle corresponding livestock 3 is, or history of breeding cycle. The estrus sign of the livestock 3 is information of a degree of the estrus sign in the unfertilized state. The fertilization information is information of a date and the number of times the fertilization is performed, and bull. The pregnancy diagnosis information is information of a date on which pregnancy diagnosis after fertilization is performed and a result of the diagnosis. Sex identification information is information of sex of fetus of pregnant cattle. The non-lactating period information is information of a schedule of a non-lactating period, and whether or not it is in the non-lactating period. The number of empty womb days is information of the number of elapsed days since childbirth. The childbirth information is information of an expected childbirth date and history of childbirth of pregnant cattle.

These pieces of individual information of the livestock 3, as information for managing livestock 3, is updated at any time by a breeder or veterinarian who manages livestock 3 inputting information to the breeder-side information terminal 50 or the veterinarian-side information terminal 70 to transmit update information to the information processing server 30.

In addition to these pieces of breeding history, various pieces of information such as medication history, injury/disease history, and bait purchase history of individual livestock 3 may be recorded in the database storage unit 330. Furthermore, the database storage unit 330 may store at least one of information of a place in which livestock is bred, information of a period during which livestock is put in different pastures and the like, and information of a surround environment such as a temperature or sunshine duration of a breeding place. These pieces of information may also be transmitted to the information processing server 30 via the communication network 40 by being input to the breeder-side information terminal 50 or the veterinarian-side information terminal 70. These pieces of information are managed mainly using a conventional paper medium, and thus inconvenience due to trouble in daily filling in, difficulty in copying, troublesomeness caused by the large number of ledgers, securing of a storage location, and the like have occurred. However, if these pieces of information are digitalized information, it is easy to be shared by not only a breeder and a veterinarian but also other related persons, and it is easy to output or transmit information.

Furthermore, information such as an estrus date, a fertilization date, an estrus starting time, an estrus ending time, an intensity of estrus, and a length of estrus duration time of each head of the livestock 3 may be recorded in the database storage unit 330 by a breeder or veterinarian. These pieces of information may be transmitted to the information processing server 30 via the communication network 40 by being input to the breeder-side information terminal 50 or the veterinarian-side information terminal 70.

### <2-3. Control Unit>

The control unit 320 may also be constituted by an arithmetic processing device such as an MPU or a CPU, and memories such as a ROM and a RAM for storing a control program, control parameters, and the like. In this case, functions of respective units included in the control unit 320 can be realized by the arithmetic processing device executing a control program stored in a memory.

### [2-3-1. Database Control Unit]

The database control unit 325 controls the individual information of the livestock 3 stored in the database storage unit 330. Specifically, the database control unit 325 adds, updates, or deletes the individual information of the livestock 3 stored in the database storage unit 330 on the basis of an input of information by a breeder or veterinarian who manages livestock 3. An input of update information or the like may be performed via the breeder-side information terminal 50 or the veterinarian-side information terminal 70.

In a case in which the signal transmission device 10A includes various sensors, the database control unit 325 may add, update, or delete the individual information of the livestock 3 on the basis of information of the state of the livestock 3 measured by various sensors, or information of the state of a surrounding environment of the livestock 3. For example, the database control unit 325 may change information of breeding such as an estrus state or a childbirth state among the individual information of the livestock 3 on the basis of temperature information or vibration information included in a signal transmitted from the signal transmission device 10A.

### [2-3-2. Signal Processing Unit]

The signal processing unit 340 executes predetermined processing mainly on the basis of a transmission signal (relay signal) transmitted from the signal transmission device 10A. In the information processing system 1 according to the present embodiment, a relay signal transmitted from the signal transmission device 10A and received via the signal relay device 10B can include identification information for identifying the signal transmission device 10A and the signal relay device 10B, sensor information such as vibration information and temperature information detected by the sensor unit 115, information indicating a time at which the signal relay device 10B has received a transmission signal, and the like. Hereinafter, a case in which the vibration information is a value of an inter-terminal voltage of a power accumulation unit that accumulates a part of vibration generated electric power generated by a piezoelectric element will be described as an example.

### (2-3-2-1. Vibration Generated Electric Power Integration Unit)

The vibration generated electric power integration unit 343 obtains an integrated value of vibration generated electric power of a signal transmission device 10A attached to each head of livestock on the basis of vibration information included in a received relay signal. The integrated value of vibration generated electric power per unit time corresponds to an activity amount of the livestock 3. For example, the vibration generated electric power integration unit 343 may obtain an integrated value of an inter-terminal voltage V indicating a vibration generated electric power amount generated by a vibration sensor in a predetermined unit period set in advance and accumulated in a power accumulation unit for vibration power generation. For example, in a case in which the signal transmission device 10A includes a solar battery as the power generation unit 111, since a generated electric power amount of the solar battery is large and a transmission signal is transmitted from the signal transmission device 10A at an appropriate frequency in a time zone in which the sun can radiate, vibration information correlated with the activity amount of the livestock 3 can be sequentially transmitted to the information processing server 30. On the other hand, since the generated electric power amount of the solar battery is small and a transmission interval of a transmission signal from the signal transmission device 10A is long in a time zone in which the sun does not radiate, the information processing server 30 may not be able to correctly ascertain vibration information in some cases.

For this reason, a time zone in which the sun can radiate, for example, 8 o'clock to 15 o'clock, is set as a unit period, and inter-terminal voltages V which are vibration information included in transmission signals transmitted during this unit period every day may be integrated. Alternatively, time from 8 o'clock to 14 o'clock is divided by every 2 hours, each period of 8 o'clock to 10 o'clock, 10 o'clock to 12 o'clock, and 12 o'clock to 14 o'clock is set as a unit period, and inter-terminal voltages V which are vibration information included in transmitted transmission signals may be integrated. A calculated voltage integrated value ∫V is stored in the database storage unit 330 by the database control unit 325.

### (2-3-2-2. Temperature Difference Calculation Unit)

The temperature difference calculation unit 345 obtains a temperature difference ΔT on the basis of a first temperature T1 and a second temperature T2 included in a received relay signal. The temperature difference ΔT between the first temperature T1 and the second temperature T2 correlates with a metabolic rate of the livestock 3. The temperature difference calculation unit 345 obtains the temperature difference ΔT between the first temperature T1 and the second temperature T2 included in each transmission signal transmitted from the signal transmission device 10A. The calculated temperature difference ΔT is stored in the database storage unit 330 by the database control unit 325.

In the information processing system 1 according to the present embodiment, the first temperature T1 is a temperature correlated with a body temperature of the livestock 3, and the second temperature T2 is a temperature correlated with the outside air temperature. A healthy livestock can include a large metabolism and a big temperature difference ΔT. The temperature difference ΔT between the first temperature T1 and the second temperature T2 included in each of received signals may be individually stored in the database storage unit 330, and for example, an integrated value ∫ΔT obtained by integrating temperature differences AT on a day basis may be stored in the database storage unit 330.

### (2-3-2-3. State Determination Unit)

The state determination unit 341 determines the state of livestock 3 on the basis of the voltage integrated value ∫V and information related to the temperature difference ΔT. For example, the voltage integrated value ∫V obtained on basis of the vibration information correlates with the activity amount of livestock 3, and the activity amount of livestock 3 correlates with an estrus state. In addition, the temperature difference ΔT obtained on the basis of temperature information correlates with the metabolism of livestock 3, and the metabolism correlates with a childbirth state. For this reason, the state determination unit 341 determines the state of livestock 3 on the basis of the voltage integrated value ∫V obtained on the basis of vibration information and information related to the temperature difference ΔT obtained on the basis of temperature information.

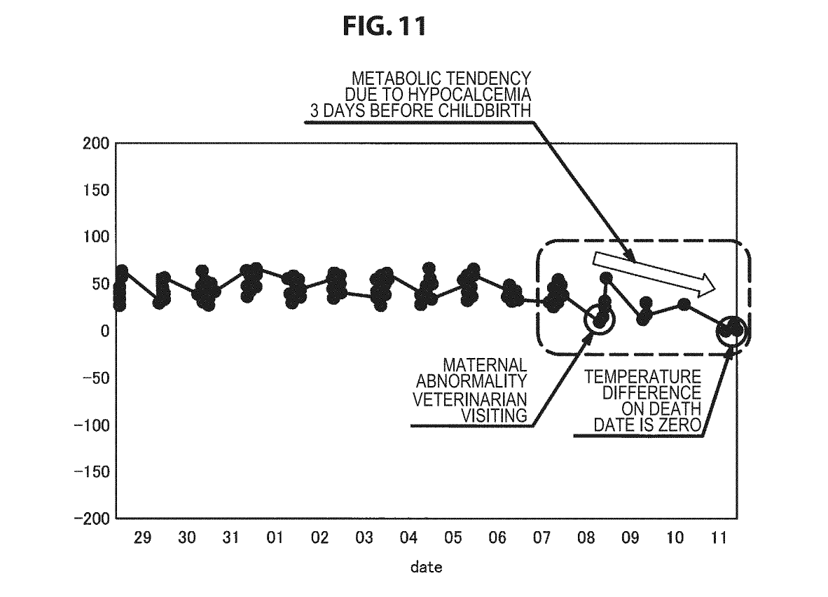

FIG. 11 shows a change in the temperature difference ΔT between a first temperature T1 detected on the body surface side of dairy cattle and a second temperature T2 detected toward a side opposite to the body surface side. A metabolic rate of dairy cattle replaced with the temperature difference ΔT and displayed shows that metabolism decreases due to hypocalcemia 3 days before childbirth, and thereafter, metabolism continues to weaken and the temperature difference ΔT becomes zero on the day of death. Therefore, it can be known that it is appropriate to regard the temperature difference ΔT between a first temperature T1 and a second temperature T2 as the metabolic rate of dairy cattle.

Determination of the state of the livestock 3 which is performed on the basis of the activity amount and metabolic rate of the livestock 3 may be, for example, a death or weakness condition, a presence or absence of estrus, a presence or absence of injury/disease, and the like. Furthermore, a fall of the signal transmission device 10A can be detected on the basis of information of the activity amount and metabolic rate of the livestock 3. Information of a determination result is stored in the database storage unit 330, and can be provided in accordance with, for example, a request from an information terminal 70 of the breeder 9A or the veterinarian 9B. As a result, the breeder 9A or veterinarian 9B can ascertain the state of each head of the livestock 3 and use it for determination of a presence or absence of the livestock 3 that requires diagnosis.

### [2-3-3. Display Information Control Unit]

The information control unit 350 mainly performs various types of control on the basis of information input to the breeder-side information terminal 50 and the veterinarian-side information terminal 70. The information control unit 350 includes a display information control unit 351, a notification control unit 353, and a learning control unit 355.

### (2-3-3-1. Display Information Control Unit)

The display information control unit 351 causes predetermined display information to be transmitted to the breeder-side information terminal 50 or the veterinarian-side information terminal 70 in accordance with a request from the breeder-side information terminal 50 or the veterinarian-side information terminal 70. For example, the display information control unit 351, in a case in which there is a request for management information from the breeder-side information terminal 50 or the veterinarian-side information terminal 70, may provide requested display information of a list of current state information of a plurality of heads of the livestock 3 bred in a pasture. The management information may be, for example, information indicating in which state of the breeding cycle shown in FIG. 10 individual livestock 3 is. With the management information, a breeder or a veterinarian can ascertain which processing will be required for corresponding livestock 3 next.

In addition, the display information control unit 351, in a case in which there is a request for breeding information from the breeder-side information terminal 50 or the veterinarian-side information terminal 70, may provide requested display information of a list of current breeding information of the livestock 3. At this time, the display information may include information of a transition of an activity amount indicating a characteristic change in an estrus period. A transition of the activity amount may be a transition of the integrated value ∫V of the inter-terminal voltages V indicating vibration generated electric power amounts generated by a piezoelectric element serving as a vibration sensor in a unit time set in advance. A transition of the activity amount may be, for example, a transition of the integrated value ∫V of the inter-terminal voltages V per day. As a result, a breeder or a veterinarian who has seen breeding information can determine the estrus of the livestock 3 in conjunction with other pieces of breeding information while viewing a transition of the activity amount. Note that the activity amount is an example of the state quantity showing a characteristic change in an estrus period, and a transition of the state quantity other than the activity amount may also be displayed. For example, a value of electrical conductivity in a vagina or a transition of a pH value and the like in the vagina may be displayed.

In addition, the display information control unit 351, when causing a transition of the activity amount to be displayed, may cause data of a reference transition to be displayed together with measurement data of the integrated value ∫V of the inter-terminal voltages V of target livestock 3. With data of a reference transition being displayed, a breeder can predict estrus by comparing measurement data with the data of a reference transition. The reference transition may be, for example, a transition of the activity amount of a last estrus cycle of corresponding livestock 3, and may also be data of an average transition of activity amounts in past estrus cycles. Alternatively, the reference transition may be data of an average transition of activity amounts in estrus cycles of a plurality of heads of the livestock 3 of the same type. An average transition of past activity amounts of corresponding livestock 3 can be generated as a learning result by the learning control unit 355.

In addition, the display information control unit 351 may cause a last estrus date to be displayed when information of a transition of the activity amount is displayed. As a result, a breeder can easily predict next estrus. In addition, the display information control unit 351 may cause a next predicted estrus date to be displayed when information of a transition of the activity amount is displayed. The predicted estrus date can be predicted as a learning result by the learning control unit 355. For example, the display information control unit 351 may predict a predicted estrus date on the basis of history of at least one type of information among the estrus date, fertilization date, estrus intensity, and estrus duration time of individual livestock 3.

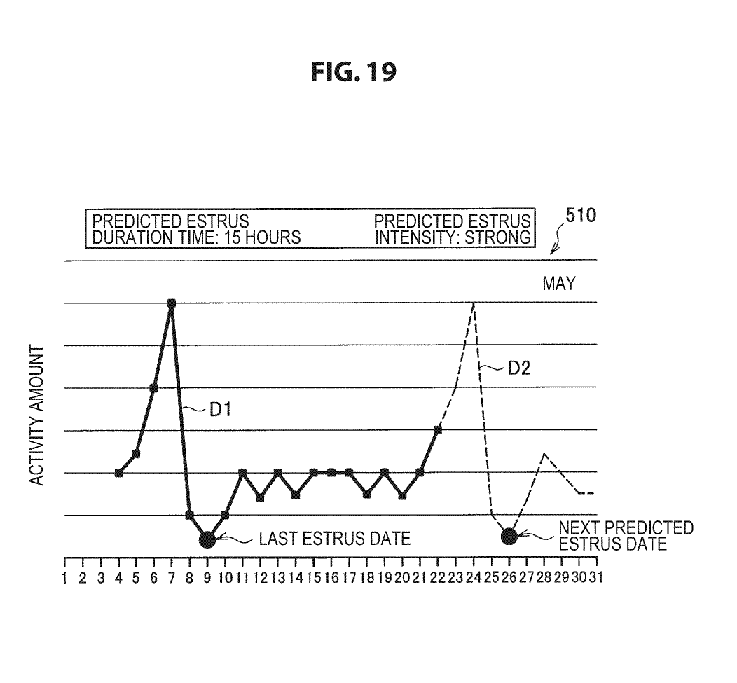

Furthermore, the display information control unit 351 may cause a predicted estrus duration time to be also displayed with reference to a past estrus duration period (refer to FIG. 19). In the same manner, the display information control unit 351 may cause a prediction estrus intensity to be also displayed with reference to a past estrus intensity (refer to FIG. 19). The predicted estrus duration time or predicted estrus intensity may be an average value in the past, or may be immediately preceding information or an average value of the latest appropriate numbers. It is possible to prevent a timing of fertilization from being missed by performing such displays together.

### (2-3-3-2. Notification Control Unit)

The notification control unit 353 notifies the veterinarian-side information terminal 70 that estrus contact has been made when an estrus notification request to a veterinarian has been input to the breeder-side information terminal 50. The notification can be performed by various methods such as an image display, a sound output, or turning on a light source. A veterinarian who has received a notification that estrus contact has been made inputs a request for breeding information to the information processing server 30 using, for example, the veterinarian-side information terminal 70, thereby causing breeding information to be displayed on the veterinarian-side information terminal 70 and checking a current state of the livestock 3.

In addition, the notification control unit 353 causes time information indicating time at which a notification request is made to be stored in the database storage unit 330 as estrus history of the livestock 3 when an estrus notification request to a veterinarian has been input to the breeder-side information terminal 50. For example, the notification control unit 353 causes a date of the day to be registered in the database storage unit 330 as an estrus date. In other words, when estrus contact is made to a veterinarian from a breeder, the estrus date is registered at the same time. A time at which the notification request is made may also be stored in the database storage unit 330 together with the estrus date. Therefore, a breeder inputs estrus contact with a veterinarian to the breeder-side information terminal 50, and thereby information of an estrus date is accumulated in the database storage unit 330 as internal processing.

The learning control unit 355 executes learning processing of a reference transition of the activity amount to be displayed together with information 510 on a transition of the activity amount on the basis of information of the estrus history of individual livestock 3 and information of history of activity amount registered in a database. For example, the learning control unit 355 may learn the reference transition of the activity amount by averaging transitions of the activity amount for each estrus cycle. In this case, a plurality of times of latest transitions of activity amount set in advance may be used for each head of the livestock 3. In addition, the transition of the activity amount for each estrus cycle of a plurality of heads of the livestock 3 may also be used. By providing the learning control unit 355, a breeder can easily assist the determination of estrus, and determine a timing and the like of fertilization.

Specifically, the learning control unit 355 may perform learning processing of generating reference data for determining estrus or abnormality of the livestock 3 using sensor information included in a transmission signal transmitted from the signal transmission device 10A, information of an estrus data input to the breeder-side information terminal 50 or the veterinarian-side information terminal 70 by a breeder or veterinarian, information of an estrus diagnosis result, information of a pregnancy diagnosis result, information of a sex identification result, information of a childbirth result, and the like. By providing such a learning control unit 355, it becomes easy to assist determination of estrus by a breeder, and to determine a timing for fertilization.

In addition, the learning control unit 355 may calculate a standard estrus cycle of corresponding livestock 3 on the basis of history of past estrus data and fertilization date of individual livestock 3. In addition, the learning control unit 355 may calculate a standard estrus duration time of corresponding livestock 3 on the basis of past estrus starting time and estrus ending time of individual livestock 3. For example, in a case I which estrus continues as a result of estrus intensity diagnosis performed by a veterinarian at the time of a fertilization operation after an operation input of notification of estrus contact from a breeder to a veterinarian is performed, time from an operation input of notification of a corresponding estrus contact to the estrus intensity diagnosis may be set as duration time. In addition, the learning control unit 355 may calculate an intensity of standard estrus of corresponding livestock 3 on the basis of a past estrus intensity of individual livestock 3. The learning control unit 355 may correct a threshold value of the activity amount for performing estrus determination by the state determination unit 341 in accordance with the intensity of standard estrus of individual livestock 3. As a result, a presence or absence of estrus is determined in consideration of an individual difference of the livestock 3.

In addition, the learning control unit 355 may calculate a standard estrus duration time of corresponding livestock 3 on the basis of a length of past estrus duration time of individual livestock 3. Furthermore, the learning control unit 355 may obtain a predicted estrus date on the basis of one or a plurality of pieces of information of a calculated standard estrus cycle, estrus duration time, and an estrus intensity. Information obtained by the learning control unit 355 may be displayed, for example, together with a transition of the activity amount included in the display information of breeding information. As a result, a breeder or a veterinarian can share various pieces of information concerning the breeding of the livestock 3. In addition, a breeder or a veterinarian can easily estimate a next estrus date of individual livestock 3. Note that the state determination unit 341 may perform state determination processing using an algorithm based on a learning result by the learning control unit 355.

### <2-4. Hardware Configuration of Information Processing Device>

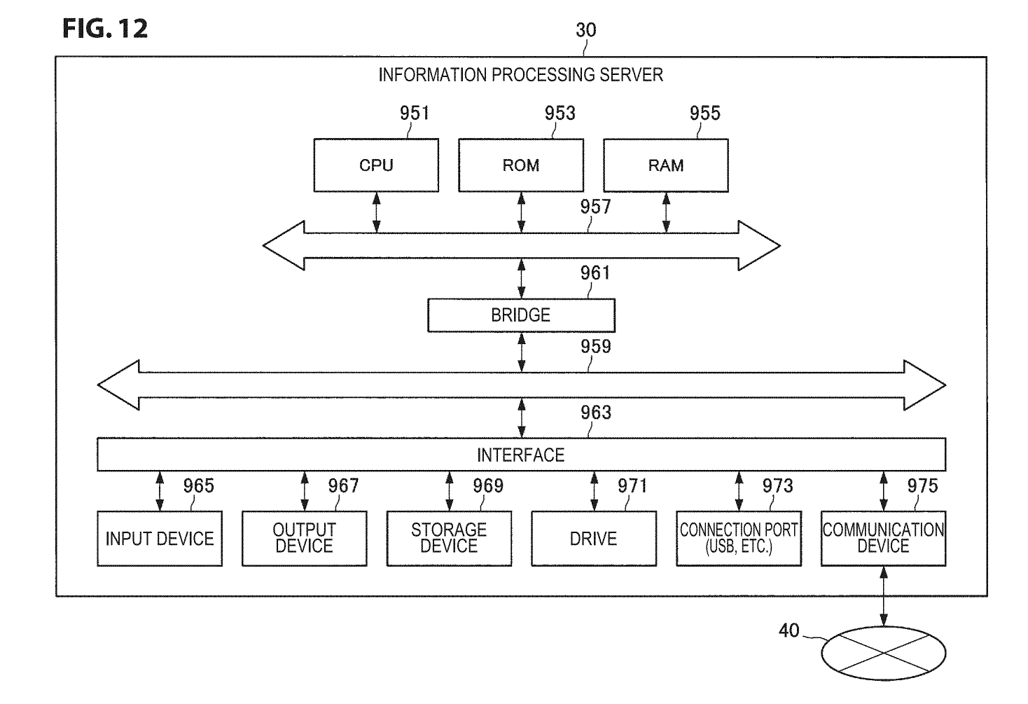

Next, a hardware configuration of the information processing device 30 according to the present embodiment will be described with reference to FIG. 12. FIG. 12 is a block diagram which shows an example of the hardware configuration of the information processing device 30 according to the embodiment. Note that information processing by the information processing device 30 according to the present embodiment is realized by cooperation between software and hardware.

As shown in FIG. 12, the information processing device 30 includes a CPU 951, a ROM 953, a RAM 955, a bridge 961, internal buses 957 and 959, an interface 963, an input device 965, an output device 967, a storage device 969, a drive 971, a connection port 973, and a communication device 975.

The CPU 951 functions as an arithmetic processing device and a control device, controls an overall operation of the information processing device 30 according to various programs stored in the ROM 953 or the like. The ROM 953 stores programs and arithmetic operation parameters used by the CPU 951, and the RAM 955 temporarily stores a program to be used in an execution of the CPU 951, parameters that appropriately change in execution thereof, and the like. For example, the CPU 951 may execute the function of the control unit 320.

The CPU 951, the ROM 953, and the RAM 955 are connected to one another using the bridge 961, the internal buses 957, 959, and the like. In addition, the CPU 951, the ROM 953, and the RAM 955 are also connected to the input device 965, the output device 967, the storage device 969, the drive 971, the connection port 973, and the communication device 975 via the interface 963.

The input device 965 includes input devices such as a touch panel, a keyboard, a mouse, a button, a microphone, a switch, a lever to which information is input. The input device 965 also includes an input control circuit for generating an input signal on the basis of the input information and outputting the input signal to the CPU 951.

The output device 967 includes a display device such as a CRT display device, a liquid crystal display device, an organic EL display device, or the like. Furthermore, the output device 967 may include a sound output device such as a speaker and a headphone.

The storage device 969 is a store device for storing data of the information processing server 30. The storage device 969 may include a storage medium, a storage device for storing data in the storage medium, a reading device for reading data from the storage medium, and a deletion device for deleting stored data. The storage device 969 may execute the function of the database storage unit 330.

The drive 971 is a read/writer for a store medium, which is incorporated in the information processing device 30 or externally attached thereto. For example, the drive 971 can read information stored in an attached removable store medium such as a magnetic disk, an optical disk, a magneto-optical disk, or a semiconductor memory, and output it to the RAM 353. Moreover, the drive 971 can also write information to a removable storage medium.

The connection port 973 is a connection interface constituted by a connection port for connecting an external connection device such as a universal serial bus (USB) port, an Ethernet (registered trademark) port, an IEEE802.11 standard port, and an optical audio terminal.

The communication device 975 is a communication interface constituted by a communication device and the like for a connection to the communication network 40. In addition, the communication device 975 may be a wired or wireless LAN compliant communication device or a cable communication device that performs wired cable communication. The communication device 975 may execute the function of the communication network connection unit 310.

In addition, a computer program for causing hardware such as the CPU, the ROM, and the RAM built in the information processing server 30 to exhibit the same functions as respective constituents of the information processing device according to the present embodiment described above can be created. In addition, a storage medium for storing the computer program is also provided.

### 3. OPERATION OF INFORMATION PROCESSING SYSTEM

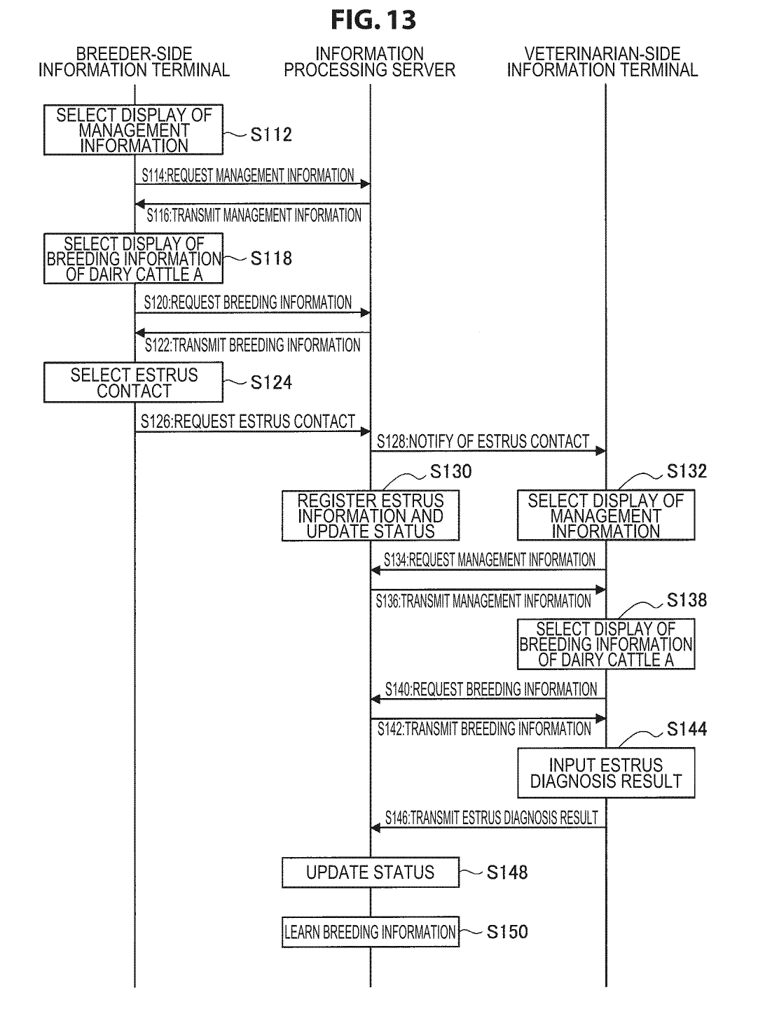

A configuration example of the information processing system 1 according to the present embodiment has been described above. Next, an example of processing by the information processing system 1 will be described with reference to FIGS. 13 to 35.

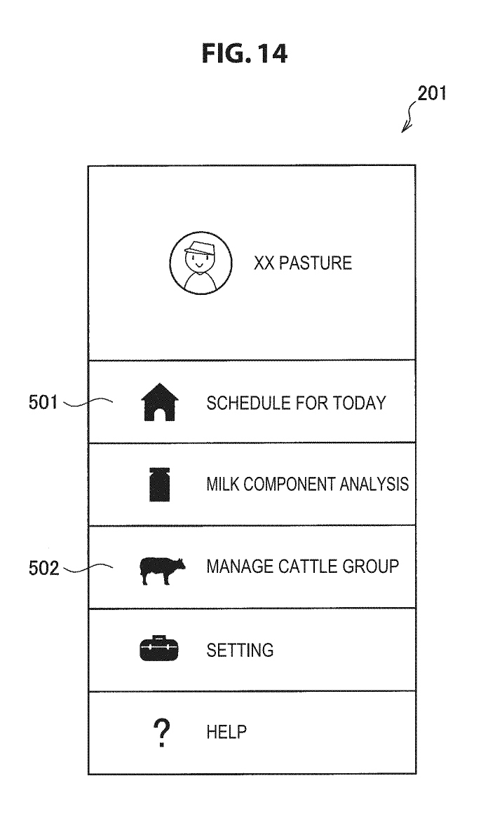

FIG. 13 is an explanatory diagram which shows a flow of processing and operations performed by the breeder-side information terminal 50, the veterinarian-side information terminal 70, and the information processing server 30 constituting the information processing system 1 in a period from the determination of estrus by a breeder to an input of an estrus diagnosis result by a veterinarian. In addition, FIGS. 14 to 26 are explanatory diagrams which show examples of images which can be displayed on the breeder-side information terminal 50 or the veterinarian-side information terminal 70 in the period. Note that images shown in FIGS. 14 to 16, and FIGS. 21 to 26 show entire images. In actual, only a range corresponding to a size of a display unit of an information terminal is displayed, and an entire image can be seen by a user (a breeder, a veterinarian, or the like) by scrolling a screen.

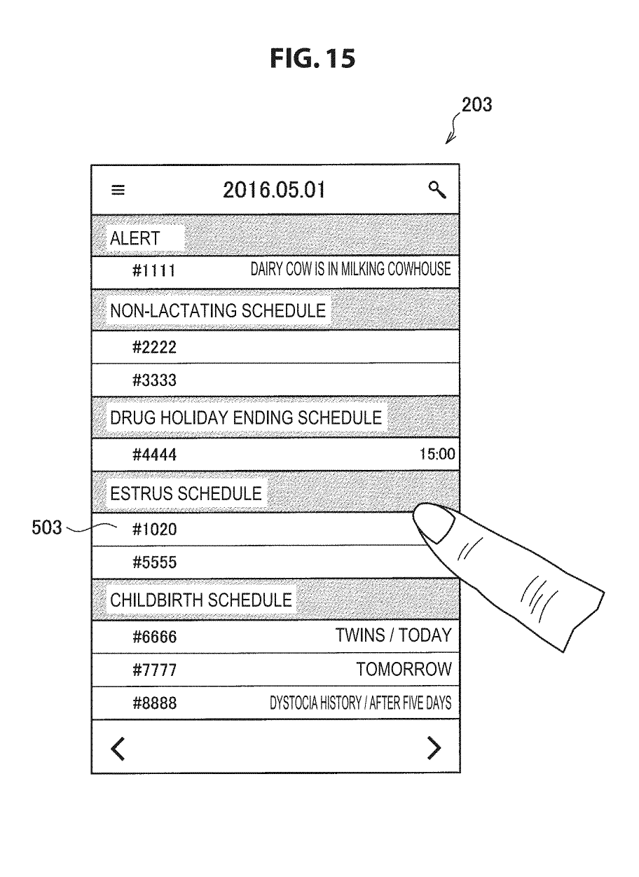

FIG. 14 shows an initial screen 201 displayed on the display unit 417 of the breeder-side information terminal 50. If the breeder 9A selects a row 501 of “today's schedule” using an operation unit 415 of the breeder-side information terminal 50, an image 203 of management information shown in FIG. 15 is displayed on the display unit 417. That is, a selection operation of a display of management information is performed by a selection of the row 501 of “today's schedule” by the breeder 9A (S112). Then, the control unit 413 of the breeder-side information terminal 50 transmits a signal requesting display information of management information to the information processing server 30 (S114).

If the information control unit 350 of the information processing server 30 receives a request for display information of management information, the display information control unit 351 transmits display information of management information in which a list of schedule of dairy cattle having some schedules today among dairy cattle bred in an XX pasture managed by the breeder 9A is summarized to the breeder-side information terminal 50 (S116). In an image 203 of the management information shown in FIG. 15, an individual identification number and a current state of each head of dairy cattle are shown. Note that dairy cattle listed in an area of “cattle scheduled for estrus” (#1020 and #5555 in an example of FIG. 15) can be identified by the control unit 320 of the information processing server 30 performing processing using the individual information, breeding information, state information, sensor information, or the like of livestock. The display information of management information includes the individual identification number of dairy cattle identified by the processing. For example, the control unit 320 can use one or a combination of the predetermined number of days having elapsed as the estrus cycle approaches since a last estrus date (for example, in a case of dairy cattle, a case in which 21 days of the estrus cycle-5 days (=16 days) has elapse), an initial sign of estrus being detected on the basis of sensor information, and the like as a determination criterion in processing at the time of identifying “cattle scheduled for estrus”. If an individual identification number of each head of dairy cattle is selected, the image 205 of the breeding information of corresponding dairy cattle is displayed on the display unit 417. Here, the breeder 9A performs an operation of selecting dairy cattle A with an individual identification number of “#1020” that is scheduled for estrus.

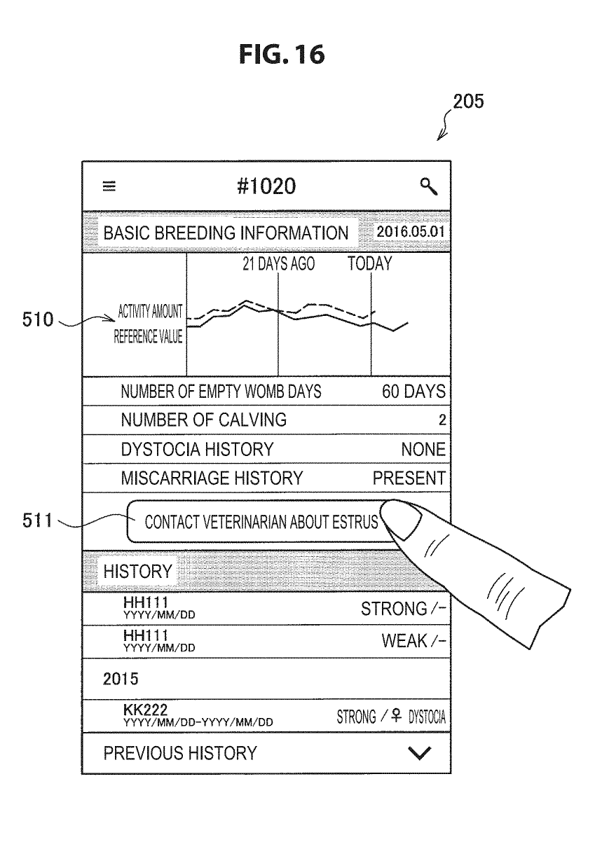

If the breeder 9A selects the dairy cattle A with an individual identification number of “#1020”, the image 205 of basic breeding information shown in FIG. 16 is displayed on the display unit 417. That is, the breeder 9A selects a row 503 of the individual identification number “#1020”, and thereby an operation of selecting a display of basic breeding information of the dairy cattle A with the individual identification number “#1020” is performed (S118). In this manner, the control unit 413 of the breeder-side information terminal 50 transmits a signal requesting the display information of the basic breeding information of dairy cattle A with the individual identification number “#1020” to the information processing server 30 (S120).

If the information control unit 350 of the information processing server 30 receives a request for the display information of the basic breeding information, the display information control unit 351 transmits the display information of the basic breeding information of dairy cattle A with the individual identification number “#1020” to the breeder-side information terminal 50 (S122). The image 205 of the basic breeding information displayed on the display unit 417 at this time includes the information 510 of a transition of the activity amount. The information 510 of a transition of the activity amount may have, for example, a point of time before at least a period of estrus cycle of livestock (21 days in the case of dairy cattle) from the present time set as a base point, and include information of a transition of the activity amount from the base point to the present time. The information of a transition of the activity amount during such a period is displayed, and thereby the breeder 9A easily predicts an estrus timing which will arrive in a few days.

In addition, the information 510 of a transition of the activity amount may include information of a reference transition of the activity amount together with information of a transition of the actual activity amount. The information of a reference transition of the activity amount is displayed together, and thereby the breeder 9A more easily predicts estrus timing. In addition to this, the display image 205 of the basic breeding information includes information of the number of empty womb days, the number of calving, dystocia history, and miscarriage history, and an estrus contact button 511 to a veterinarian as a graphical user interface (GUI) part for performing a notification to a veterinarian. In addition, the display image 205 of the basic breeding information includes a history of past breeding. The number of empty womb days is the number of days having elapsed since last childbirth. The number of calving is the number of times of childbirth in the past. Moreover, in the history of past breeding, a last estrus date, an identification number of a bull for fertilization, an intensity of estrus, a result of pregnancy diagnosis, and a childbirth result are displayed.

In the information 510 of a transition of the activity amount, the information of a transition of the actual activity amount based on vibration information detected by the vibration sensor and a reference value (reference transition) are displayed in a superimposed manner. Therefore, a breeder can determine the presence or absence of estrus depending on the presence or absence of characteristic change seen in an estrus period while observing the information 510 of a transition of the activity amount.

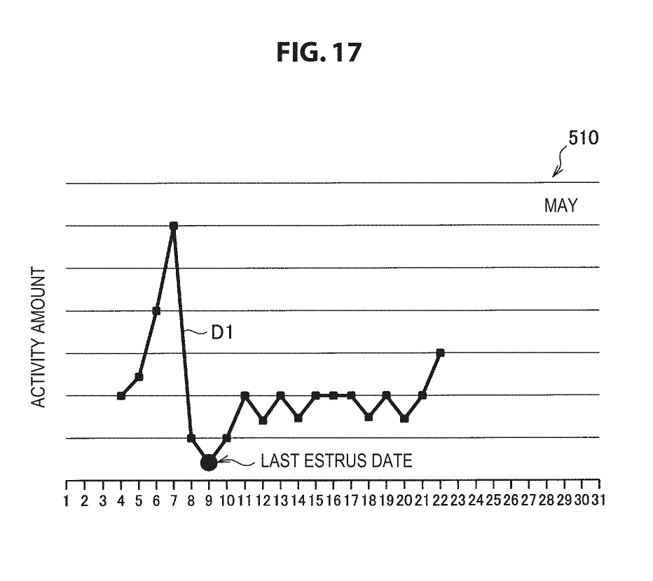

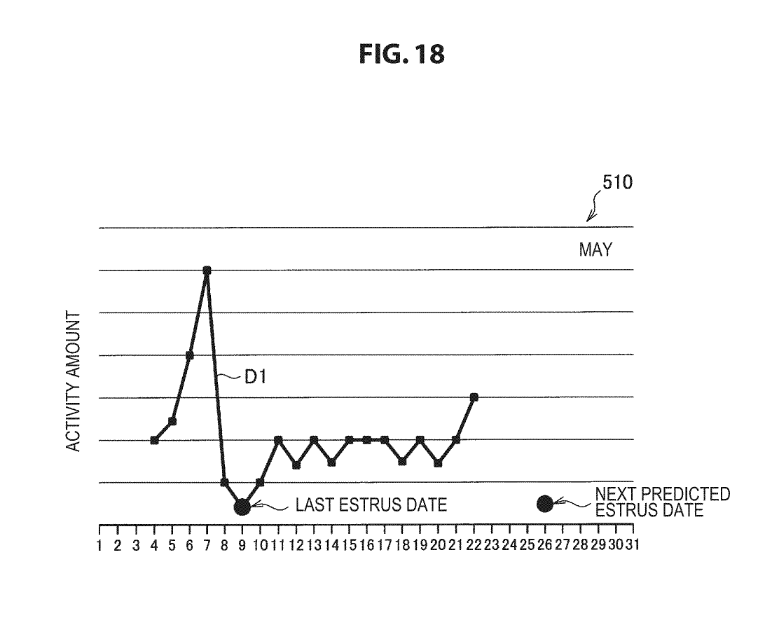

Here, a display example of the information 510 of a transition of the activity amount will be described with reference to FIGS. 17 to 20. In the display example of FIG. 17, a transition D1 of the activity amount from 5 days before a last estrus date is shown together with the last estrus date. As information of the last estrus date, information registered in the database storage unit 330 is used. In this case, since it is seen that the activity amount tends to greatly increase from 5 days before an estrus date, and the activity amount tends to sharply decrease from 2 days before the estrus date, a breeder can predict the estrus date as seeing the same tendency in few days. Note that, although not shown in FIG. 17, as shown in FIG. 16, the information 510 of a transition of the activity amount may include a display of “today” and a display of an estrus cycle of livestock (21 days in the case of dairy cattle) ago from the present time (for example, a display of “21 days ago”). The same applies to the following description in FIGS. 18 to 20.

In a display example of FIG. 18, a next predicted estrus date is further shown together with the transition amount D1 of the activity amount from 5 days before the last estrus date and the display of the last estrus date. The next predicted estrus date can be predicted by learning an average estrus cycle of the dairy cattle by, for example, the learning control unit 355. In this display example, since the predicted estrus date is visually recognized, a breeder can predict the estrus date by viewing the transition of the activity amount from about 5 days before the predicted estrus date.

In a display example of FIG. 19, a predicted transition D2 of the activity amount is further shown together with the transition amount D1 of the activity amount from 5 days before the previous estrus date, the display of the last estrus date, the display of the last estrus date, and a next predicted estrus date. The predicted transition D2 of the activity amount can be an average of transitions of the activity amount in the past estrus cycles of the dairy cattle, which is obtained by the learning control unit 355. In this display example, since the predicted transition D2 of the activity amount is further visually recognized, a breeder can easily predict an estrus date by checking that the transition D1 of the actual activity amount approximates the predicted transition D2 and transits. Moreover, in the display example of FIG. 19, a predicted estrus duration time and predicted estrus intensity are further displayed. As a result, a breeder can ascertain an optimal timing of fertilization. In the display example of FIG. 19, the display of the next predicted estrus date may be omitted.

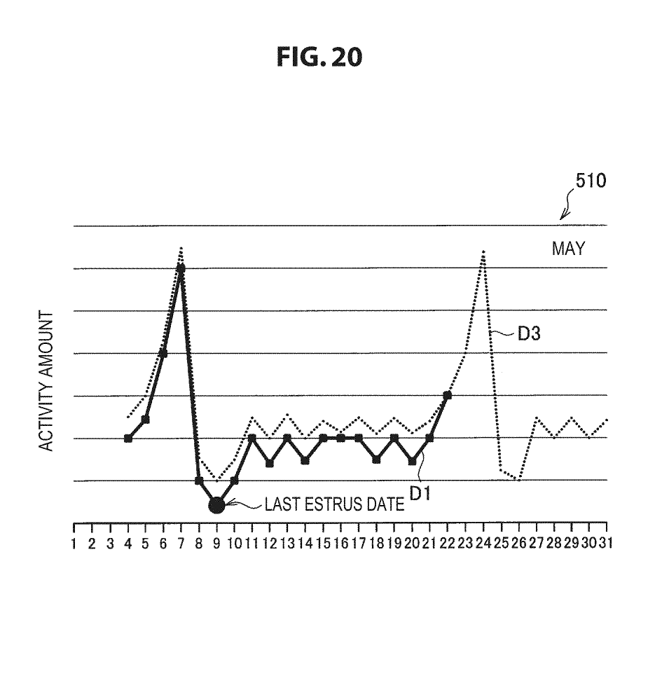

In a display example of FIG. 20, an average transition D3 of the activity amount in an estrus cycle of general dairy cattle is shown as the reference transition together with the transition amount D1 of the activity amount from 5 days before the last estrus date and the display of the last estrus date. The average transition D3 of the activity amount of the general dairy cattle can be stored in the database storage unit 330 or the like in advance. In this display example, estrus date can be predicted while comparing with the average transition D3 of the activity amount of general dairy cattle. In the display example of FIG. 20, the next predicted estrus date may be displayed together.

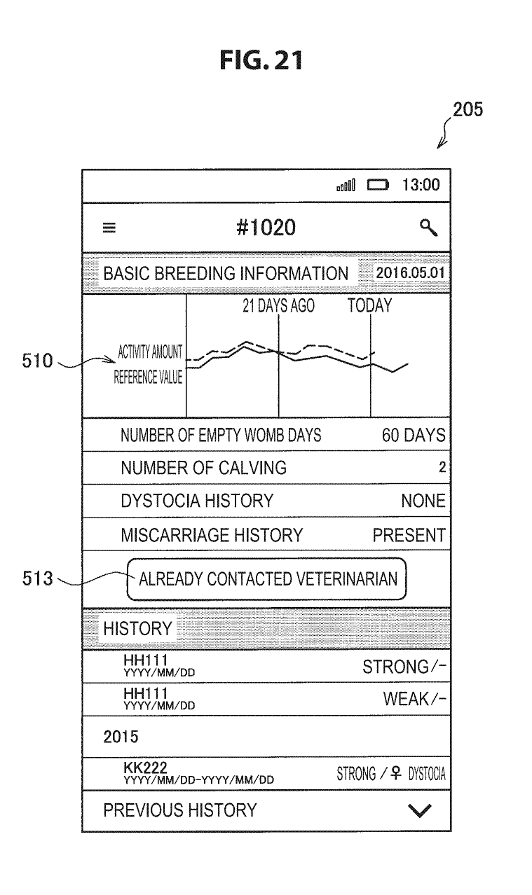

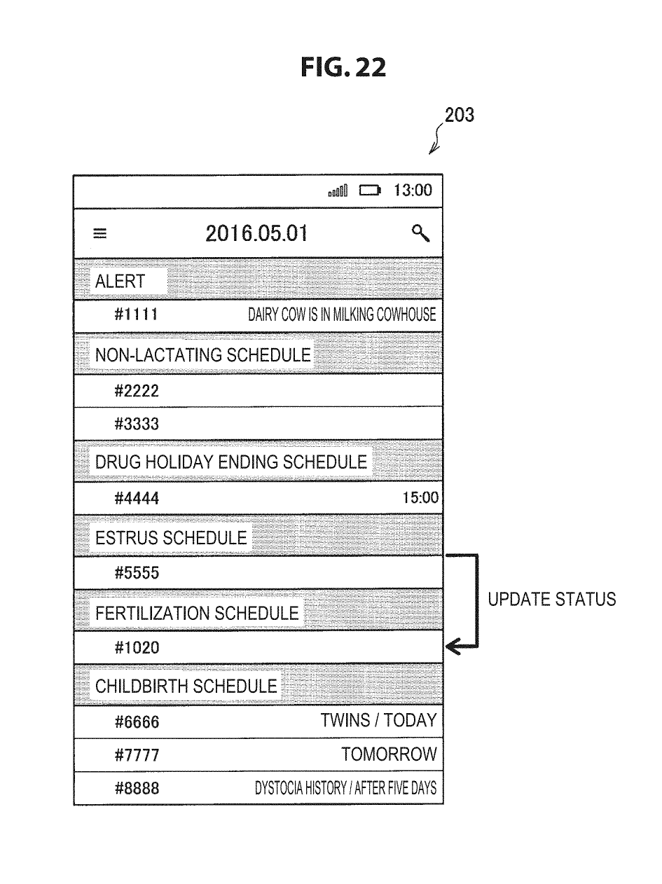

Returning to the image 205 of the basic breeding information in FIG. 16, in a case in which a breeder determines that the estrus of dairy cattle has arrived while overlooking the information 510 of a transition of the activity amount, the breeder selects the estrus contact button 511 to a veterinarian (S124). As a result, the control unit 413 of the breeder-side information terminal 50 transmits a signal of estrus contact request to the veterinarian to the information processing server 30. As shown in FIG. 21, in a case in which the estrus contact button 511 to a veterinarian is selected, the estrus contact button 511 in the image 205 of the basic breeding information is switched to a display 513 of “veterinarian contacted”. In this case, as shown in FIG. 22, the state of dairy cattle A in the image 203 of management information shifts from “estrus schedule” to “fertilization schedule.”

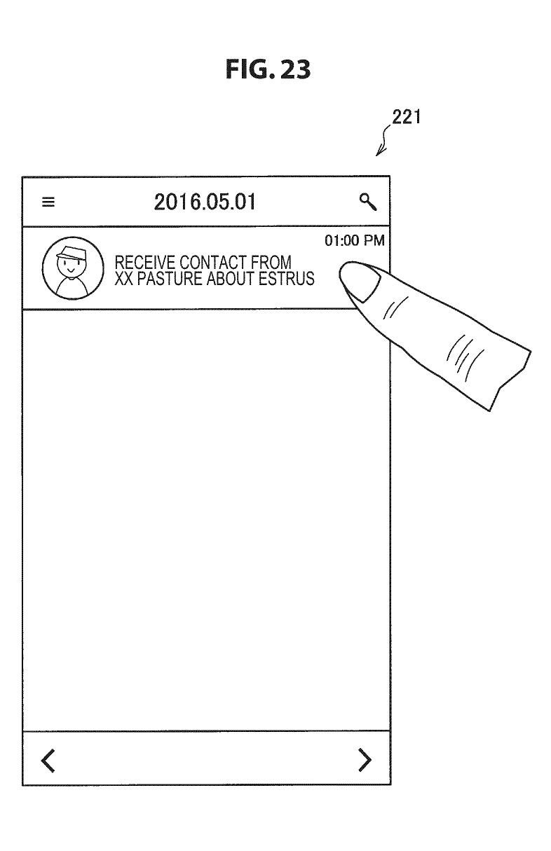

The notification control unit 353 of information processing server 30 which has received the signal of estrus contact request to a veterinarian transmits a signal of estrus contact notification to the veterinarian-side information terminal 70 (S128). At this time, as shown in FIG. 23, a notification image 221 of “estrus contact from XX pasture” is displayed in the veterinarian-side information terminal 70. At the same time, the notification control unit 353 of the information processing server 30 registers the date of receiving the signal of estrus contact request in the database storage unit 330 as the estrus date. In this manner, only with the selection operation of the estrus contact button 511 by a user (a breeder), contact with a veterinarian and registration of an estrus date using the information processing server 30 are performed.

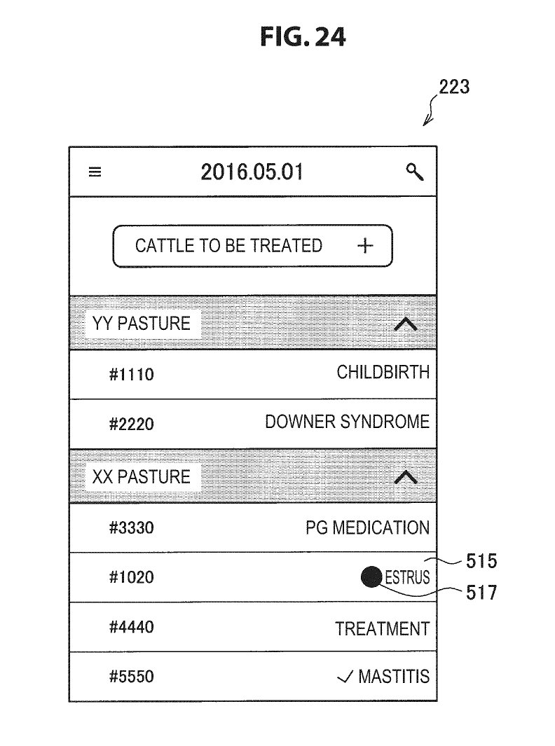

If the veterinarian 9B who has received a notification of estrus contact selects the notification, an image 223 of management information for a veterinarian is displayed as shown in FIG. 24. That is, a display of management information is selected by selecting a notification (S132). As a result, the control unit 423 of the veterinarian-side information terminal 70 transmits a signal requesting the display of management information to the information processing server 30 (S134). If information control unit 350 of the information processing server 30 receives the signal requesting the display of management information, the display information control unit 351 transmits display information of management information to the veterinarian-side information terminal 70 (S136).

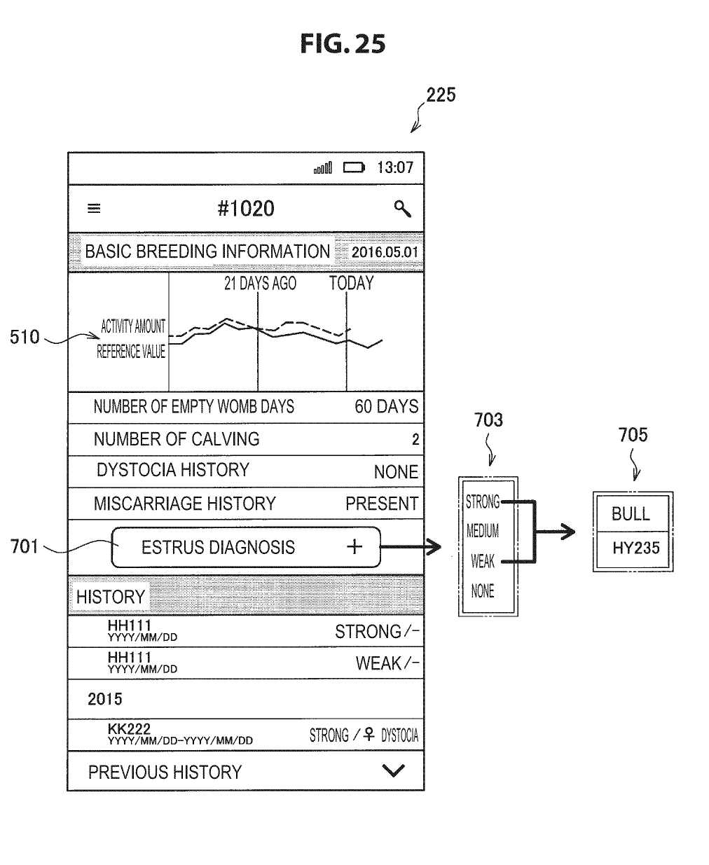

In the image 223 of the management information shown in FIG. 24, a list of information of dairy cattle undergoing diagnosis and treatment is displayed for each pasture. Here, since estrus contact is received from a breeder, a row 515 of dairy cattle with the individual identification number “#1020” shows a display of “estrus” and a display of a marking 517 indicating that a notification has been received. If a veterinarian selects the row 515 of the individual identification number “#1020”, an image 225 of the basic breeding information is displayed on display unit 427 of the veterinarian-side information terminal 70 as shown in FIG. 25. That is, by selecting the row 515 of the individual identification number “#1020”, the display of the basic breeding information of dairy cattle A is selected (S138). As a result, the control unit 423 of the veterinarian-side information terminal 70 transmits the signal requesting display information of the basic breeding information of dairy cattle A to the information processing server 30 (S140).

If the information control unit 350 of the information processing server 30 receives the signal requesting the display information of the basic breeding information, the display information control unit 351 transmits the display information of the basic breeding information to the veterinarian-side information terminal 70 (S142). The display information of the basic breeding information shown in FIG. 25 is basically the same display as the basic breeding information displayed on the breeder-side information terminal 50, but an “estrus diagnosis” button 701 is included in the image 225 of the basic breeding information of the veterinarian-side information terminal 70. Since an estrus diagnosis needs to be performed by a veterinarian, the “estrus diagnosis” button is not displayed on the breeder-side information terminal 50.

The veterinarian actually goes to a pasture, performs an estrus diagnosis on dairy cattle A, and causes the dairy cattle A to be fertilized by a bull in a case in which the veterinarian can confirm that the dairy cattle A is in the estrus state. After performing fertilization, the veterinarian selects the estrus diagnosis button 701 of the image 225 of the basic breeding information, selects an intensity of estrus on an input screen 703 of the estrus intensity result (select one of strong, medium, weak, and none), and further performs registration of a bull on a bull registration screen 705 (S144). As a result, the control unit 423 of the veterinarian-side information terminal 70 transmits information of the estrus diagnosis result to the information processing server 30 (S146). The information control unit 350 of the information processing server 30 updates the state of dairy cattle A if information of the estrus diagnosis result is received (S148). In addition, the learning control unit 355 of the information processing server 30 learns information of an average transition of activity amount and information of an estrus cycle in consideration of information of the transition of the activity amount and the estrus date in the estrus cycle this time (S150). Contents learned by the learning control unit 355 are reflected to subsequent processing of the information processing server 30. That is, when each type of processing shown in FIG. 13 is executed, information of an average transition or a reference transition of the activity amount and management information updated according to a learning result by the learning control unit 355, and breeding information are transmitted to the breeder-side information terminal 50 and the veterinarian-side information terminal 70 by the information processing server 30.

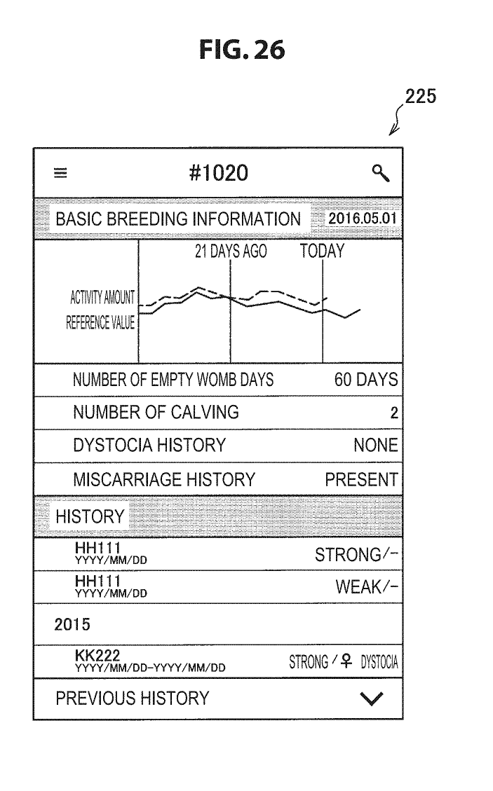

Note that, in a case in which a veterinarian has diagnosed that livestock is not in the estrus state as a result of an estrus diagnosis, as shown in FIG. 26, a button of “estrus diagnosis” disappears from the image 225 of basic breeding information.

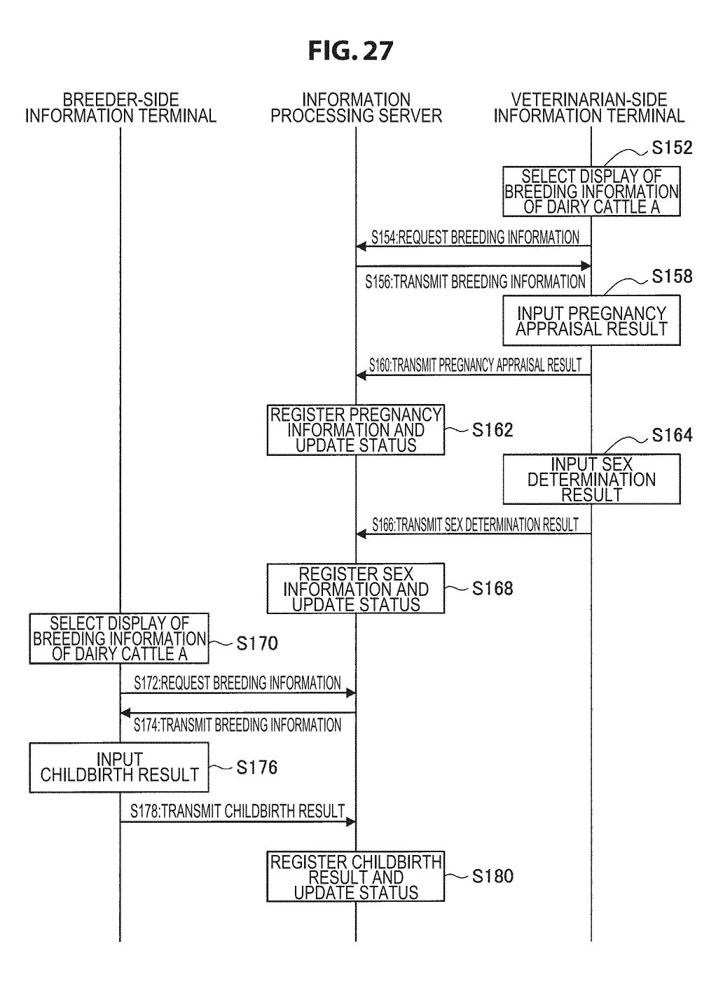

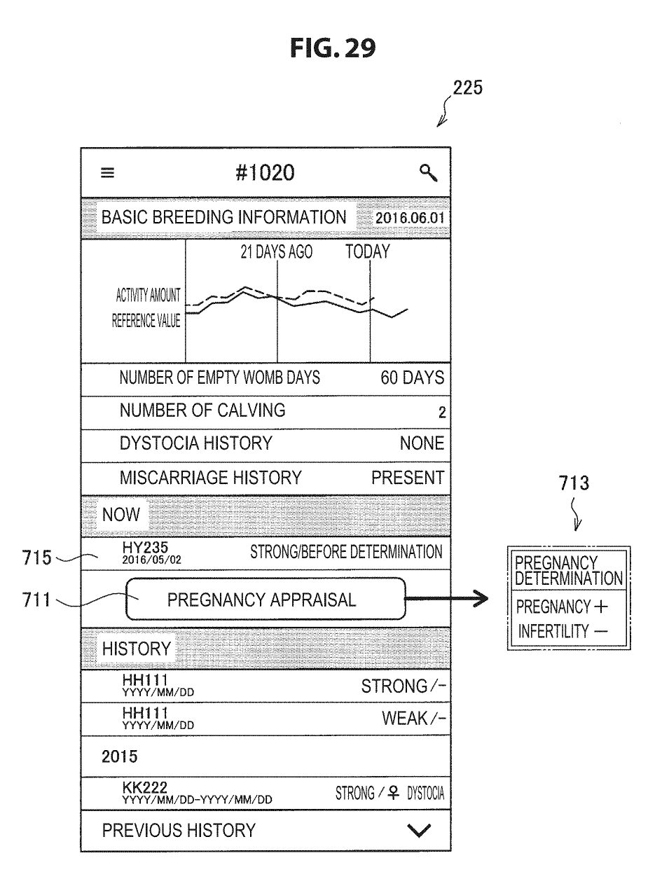

FIG. 27 is an explanatory diagram which shows a flow of processing and operations performed by the breeder-side information terminal 50, the veterinarian-side information terminal 70, and the information processing server 30 constituting the information processing system 1 in a period from a state in which dairy cattle A with the individual identification number “#1020” is fertilized to an end of childbirth. In addition, FIGS. 29 to 33 are explanatory diagrams which show examples of images that can be displayed on the breeder-side information terminal 50 or the veterinarian-side information terminal 70 in the period. Note that the images shown in FIGS. 29 to 33 show entire images, and actually only a range corresponding to a size of a display unit of an information terminal is displayed, and a user (a breeder, a veterinarian, and the like) can visually recognize the entire images by scrolling a screen.

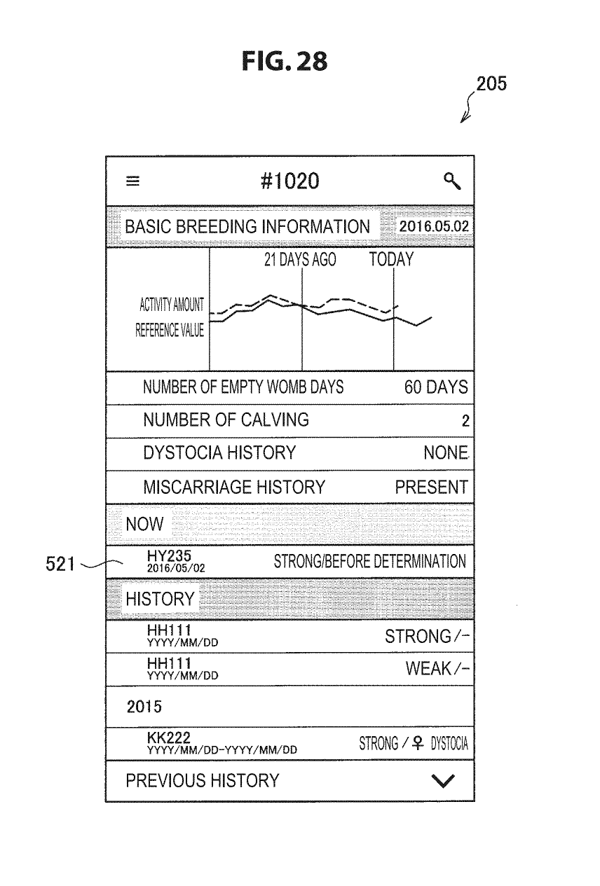

FIG. 28 shows the image 205 of the basic breeding information which can be displayed on the breeder-side information terminal 50 after the fertilization of the dairy cattle A is finished. FIG. 29 shows the image 225 of the basic breeding information which can be displayed on the veterinarian-side information terminal 70 after the fertilization of the dairy cattle A is finished. The same information is displayed on the breeder-side information terminal 50 and the veterinarian-side information terminal 70 except for a “pregnancy diagnosis” button 711 being displayed in the image 225 of the basic breeding information displayed on the veterinarian-side information terminal 70 because a pregnancy diagnosis is performed by a veterinarian after the fertilization. That is, a notation of an individual identification number “HY235” of a bull, a notation of estrus diagnosis result input date “2016.05.14”, a notation of an estrus intensity “Strong” and a notation of being in front of a pregnancy diagnosis are made in rows 521 and 715 displaying a current state of the dairy cattle A, and the “pregnancy diagnosis” button 711 is displayed only on the veterinarian-side information terminal 70.

The veterinarian selects a row of the dairy cattle A from the image 223 of management information displayed on the veterinarian-side information terminal 70 after going to a pasture and performing a pregnancy diagnosis, and thereby a display of the basic breeding information of the dairy cattle A is selected (S152). As a result, the control unit 423 of the veterinarian-side information terminal 70 transmits a signal requesting the display information of the basic breeding information to the information processing server 30 (S154). If the information control unit 350 of the information processing server 30 receives the signal requesting the display information of the basic breeding information, the display information control unit 351 transmits the display information of the basic breeding information to the veterinarian-side information terminal 70 (S156). As a result, the image 225 of the basic breeding information shown in FIG. 29 is displayed on the display unit 427 of the veterinarian-side information terminal 70.

The veterinarian selects the “pregnancy diagnosis” button 711, and selects “pregnancy+” or “infertility−” on the input screen 713 of a pregnancy determination result (S158). As a result, the control unit 423 of the veterinarian-side information terminal 70 transmits information of a pregnancy diagnosis result to the information processing server 30 (S160). The information control unit 350 of the information processing server 30, if receiving the information of a pregnancy diagnosis result, registers pregnancy information in the database storage unit 330, and updates a status of the dairy cattle A (S162).

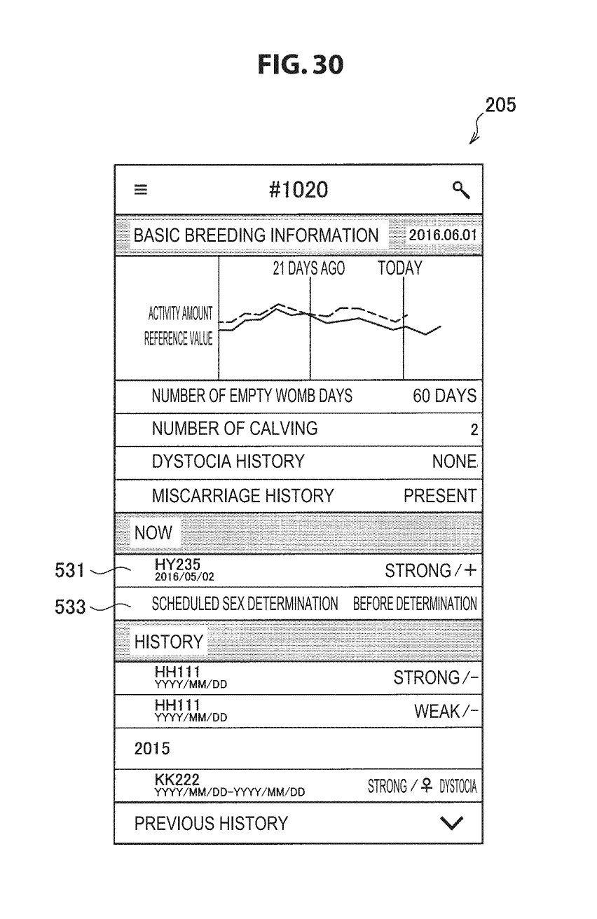

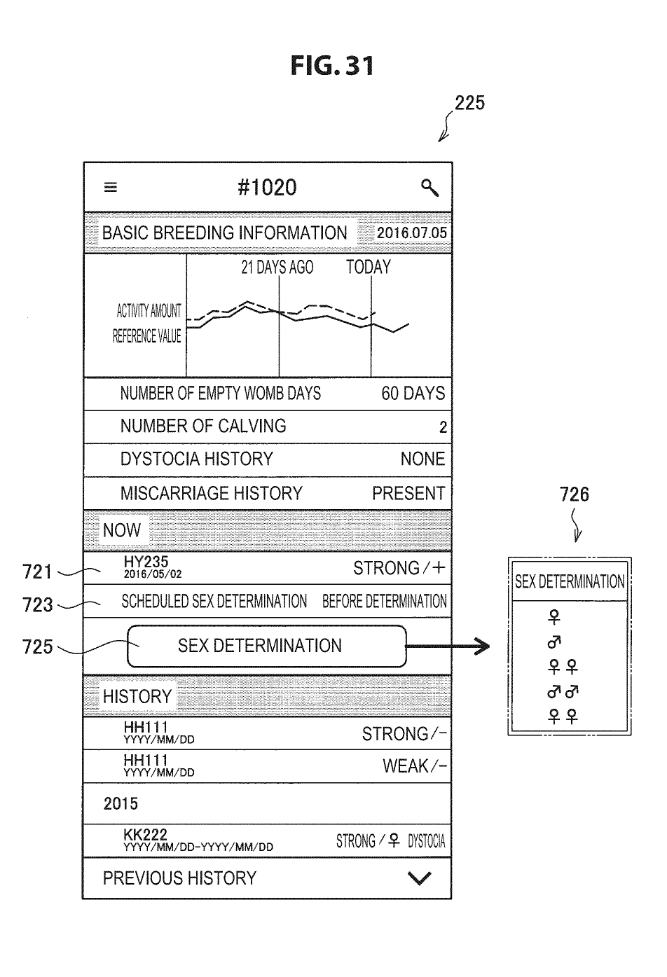

FIG. 30 shows the image 205 of the basic breeding information which can be displayed on the breeder-side information terminal 50 after the pregnancy diagnosis is finished. FIG. 31 shows the image 225 of the basic breeding information which can be displayed on the veterinarian-side information terminal 70 after the pregnancy diagnosis is finished. The same information is displayed on the breeder-side information terminal 50 and the veterinarian-side information terminal 70 except for a “sex identification” button 725 being displayed in the image 225 of the basic breeding information displayed on the veterinarian-side information terminal 70 because a veterinarian performs sex identification after pregnancy. That is, the notation of an individual identification number “HY235” of a bull, the notation of estrus diagnosis result input date “2016.05.14”, the notation of an estrus intensity “Strong”, and the notation (+) of being pregnancy are made in rows 531 and 721 displaying a current state of the dairy cattle A, and a notation of “before identification” is made in rows 533 and 723 of a scheduled sex identification. The “sex identification” button 725 is displayed only on the veterinarian-side information terminal 70.

The veterinarian selects the “sex identification” button 725 in the image 225 of the basic breeding information after going to a pasture and performing a sex identification examination, and selects a sex and the number of heads on the input screen 726 of a result of the sex identification (S164). As a result, the control unit 423 of the veterinarian-side information terminal 70 transmits information indicating a result of the sex identification to the information processing server 30 (S166). The information control unit 350 of the information processing server 30 registers information of sex identification in the database storage unit 330 and updates the status of the dairy cattle A if information indicating a result of the sex identification is received (S168).

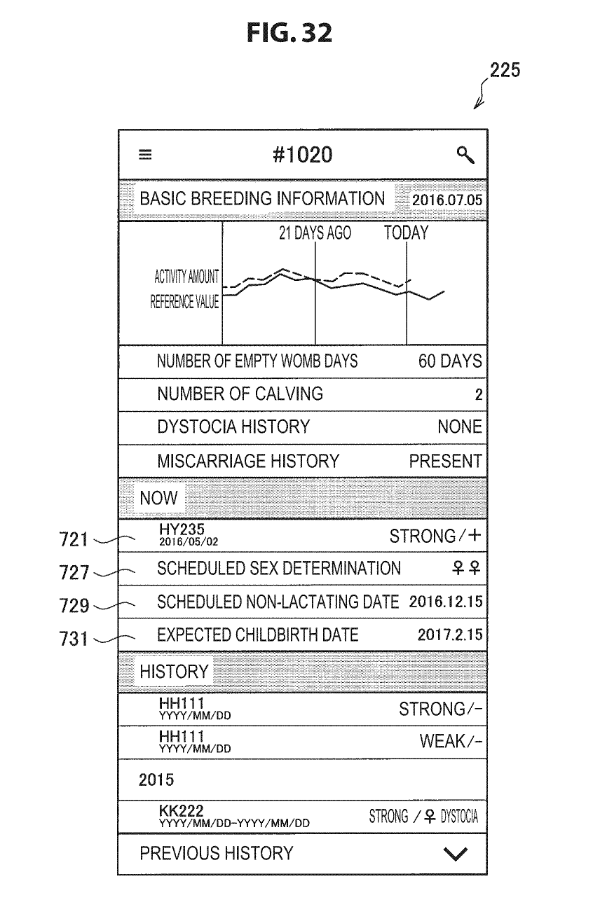

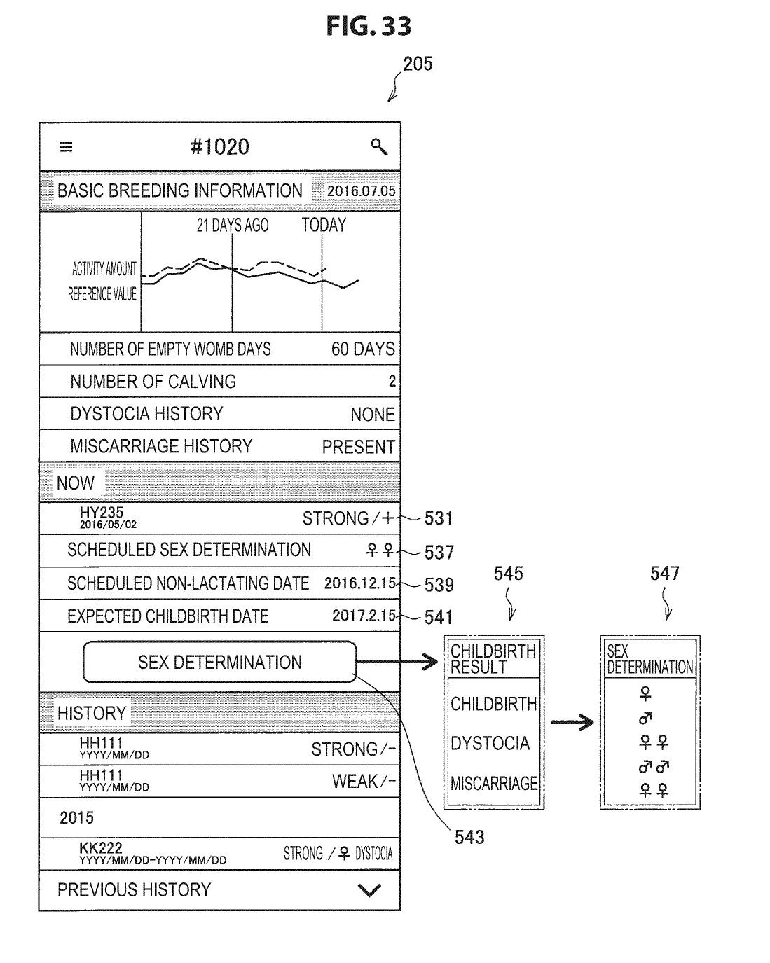

FIG. 32 shows the image 225 of the basic breeding information which can be displayed on the veterinarian-side information terminal 70 after the sex identification is finished. FIG. 33 shows the image 205 of the basic breeding information which can be displayed on the breeder-side information terminal 50 after the sex identification is finished. The same information is displayed on the breeder-side information terminal 50 and the veterinarian-side information terminal 70 except for a “childbirth result” button 543 being displayed in the image 205 of the basic breeding information displayed on the breeder-side information terminal 50 because the breeder only waits for a childbirth after the sex identification.

That is, the notation of an individual identification number “HY235” of a bull, the notation of estrus diagnosis result input date “2016.05.14”, the notation of an estrus intensity “Strong”, and the notation (+) of being pregnancy are made in rows 531 and 721 displaying a current state of the dairy cattle A, and a notation of being pregnant with two males is made in rows 537 and 727 of scheduled sex identification. In addition, schedule dates are written in rows of 539 and 729 of scheduled non-lactating date and rows 541 and 731 of expected childbirth date, respectively.

In a case in which childbirth is finished, the breeder selects a row of the dairy cattle A from the image 203 of management information displayed on the breeder-side information terminal 50, and thereby the display of the basic breeding information of the dairy cattle A is selected (S170). As a result, the control unit 413 of the breeder-side information terminal 50 transmits a signal requesting the display information of the basic breeding information to the information processing server 30 (S172). If the information control unit 350 of the information processing server 30 receives the signal requesting the display information of the basic breeding information, the display information control unit 351 transmits the display information of the basic breeding information to the breeder-side information terminal 50 (S174). As a result, the image 205 of the basic breeding information shown in FIG. 33 is displayed on the display unit 427 of the breeder-side information terminal 50.

The breeder selects the “childbirth result” button 543, selects one of childbirth, dystocia, and abortion from the input screen 545 of a childbirth result, and selects a corresponding item from a selection screen 547 for sexes and the number of heads (S176). As a result, information of a childbirth result is transmitted to the information processing server 30 (S178). The information control unit 350 of the information processing server 30, if information of a childbirth result is received, registers the information of a childbirth result in the database storage unit 330, and updates the status of the dairy cattle A (S180).

As described above, in the information processing system 1 according to the present embodiment, a breeder and a veterinarian easily update these pieces of information while sharing the individual information and breeding information of livestock (dairy cattle) 3. In addition, a display of a transition of the state quantity showing a characteristic change in the estrus period is included in the breeding information, and a breeder can determine the estrus of the livestock 3 while overlooking the display.

### 4. POSITIVE LIST INFORMATION

Configuration and operation examples of the information processing system 1 configured to mainly determine the estrus of the livestock 3 have been described. Hereinafter, an example of a utilizing method of information stored in the database storage unit 330 of the information processing server 30 will be described.

As described above, medication history, breeding history, injury/disease history, and bait purchase history may be stored in the database storage unit 330. These pieces of history information are obtained by digitizing and saving the positive list information managed using a conventional paper medium. If the positive list information is digitized, there is no inconvenience caused by daily filling in or copying by hand, and troublesomeness caused by ledger management and a storage location can be solved. Note that the positive list information shown herein may also be used for learning processing by the learning control unit 355.

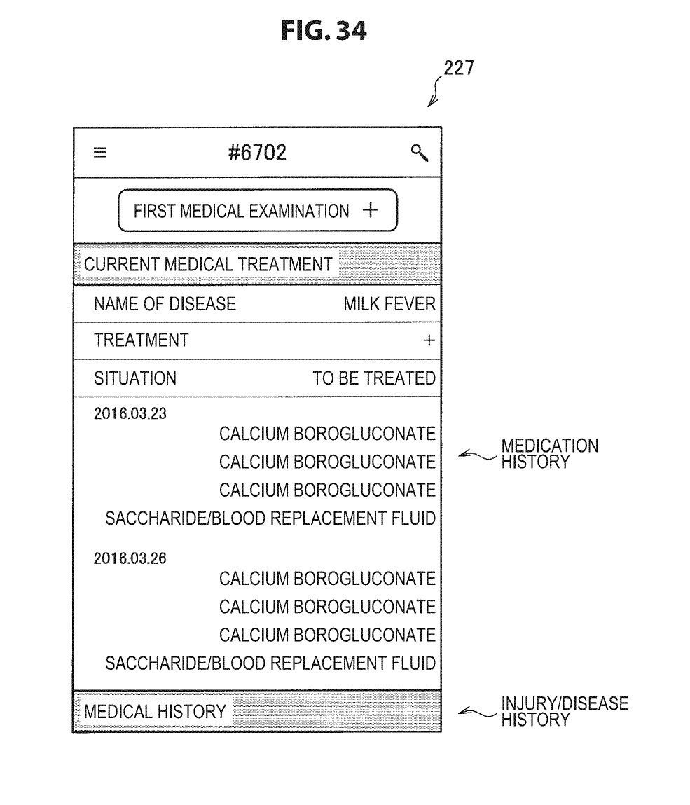

FIG. 34 shows an example of a display screen of medication history and injury/disease history. For example, a veterinarian input a name of disease, whether or not a treatment is finished (+ or −), a medication date, and a type of medicine whenever medication is performed. An injury/disease currently being treated is input to an item of “current medical treatment”, and is accumulated in an item of “medical history” when the treatment is completed. The display screen of the medication history and the injury/disease history may be displayed, for example, by selecting an item 502 of “cattle group management” in an initial screen of the breeder-side information terminal 50 shown in FIG. 14 and selecting an individual identification number of dairy cattle to be observed.

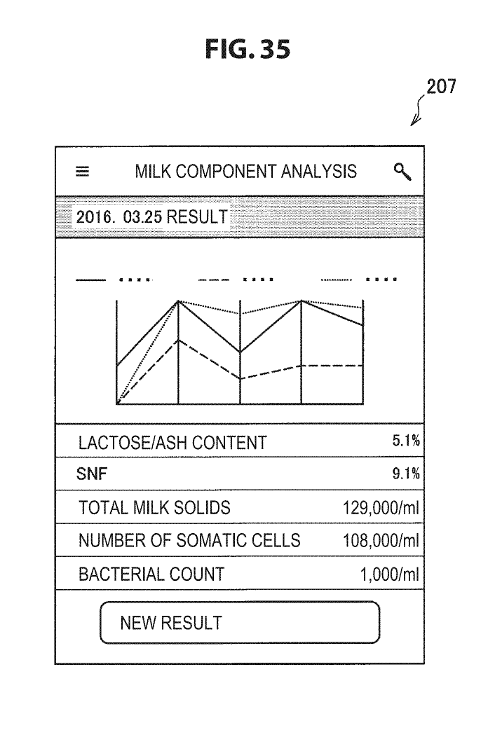

FIG. 35 shows an example of a display screen of milk component analysis. The milk component analysis is performed on the basis of various pieces of information such as bait purchase history, a milking method, breeding history, medication history, or injury/disease history. If the milk component analysis information can be simply shared or created between breeders, management of the safety of milk becomes relatively easy, and it is possible to ship out high quality raw milk.

### 5. STATE DETERMINATION METHOD

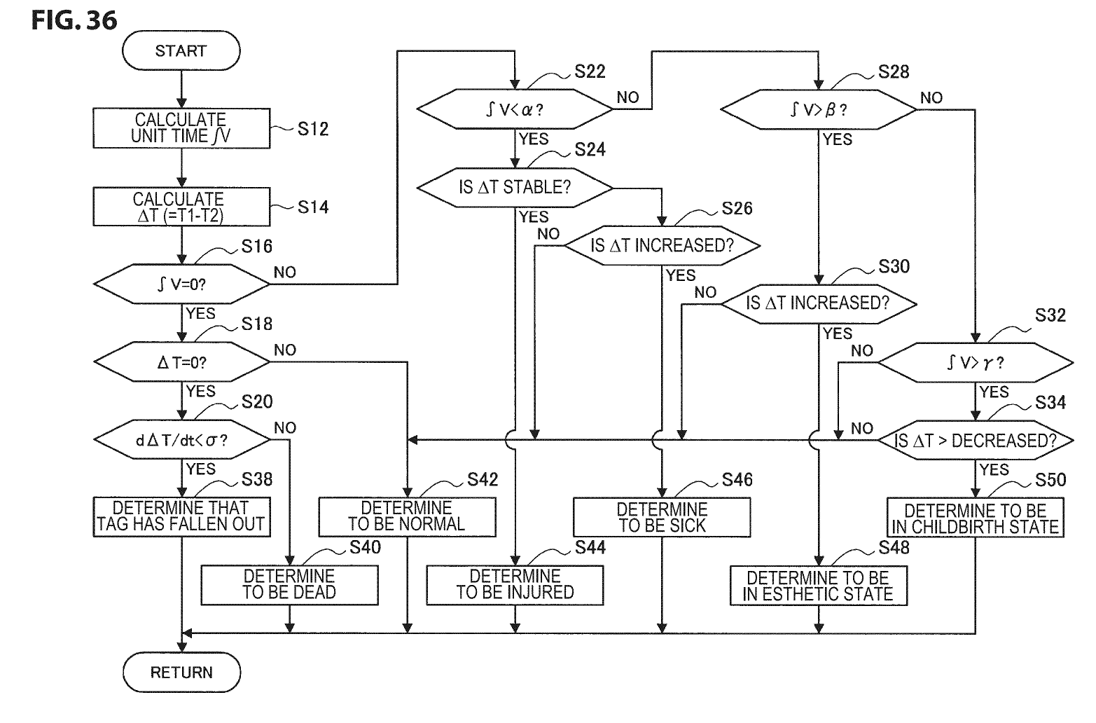

The state determination unit 341 of the information processing server 30 according to the present embodiment may determine the state of the livestock 3 on the basis of information of an integrated value ∫V of vibration generated electric power (inter-terminal voltages of power accumulation unit) correlated with the activity amount of the livestock 3 in a unit time and a temperature difference ΔT between a first temperature T1 and a second temperature T2. Hereinafter, a flowchart of the state determination processing will be described with reference to FIG. 36.

First, the vibration generated electric power integration unit 343 obtains the voltage integrated value ∫V at every unit period set in advance (S12). For example, the vibration generated electric power integration unit 343 may integrate values of the inter-terminal voltage V of the vibration power accumulation unit included in transmission signals transmitted in the daytime time zone in which transmission signals can be timely transmitted from the signal transmission device 10A attached to livestock. Next, the temperature difference calculation unit 345 obtains the temperature difference ΔT between a first temperature T1 and a second temperature T2 included in each signal (S14). For example, the temperature difference calculation unit 345 may set an average value of the absolute values of differences between a first temperature T1 and a second temperature T2 per day as the temperature difference ΔT.

Next, the state determination unit 341 determines whether or not the voltage integrated value ∫V is zero (S16). In a case in which the voltage integrated value ∫V is zero (Yes in S16), the state determination unit 341 determines whether or not the temperature difference ΔT is zero (S18). A value used for comparison with the temperature difference ΔT may be a value obtained by adding a predetermined error range to zero. In a case in which the temperature difference ΔT is zero (Yes in S18), the state determination unit 341 determines whether or not a time differentiated value dΔT/dt of the temperature difference ΔT is less than a predetermined threshold value Δ on the basis of a history of a change in the accumulated temperature difference ΔT (S20). For example, in a case in which an average value of the temperature difference per day is assumed to be the temperature difference ΔT, an amount of change in the temperature difference ΔT per day is compared with the threshold value Δ from three to four days before the temperature difference ΔT becomes zero. The threshold value Δ can be set to an appropriate value as a threshold value for determining whether the temperature difference ΔT becomes zero as a result of gradual decrease or suddenly becomes zero.

In a case in which a time differentiated value dΔT/dt of the temperature difference ΔT is less than the threshold value Δ (Yes in S20), the temperature difference ΔT becomes zero as a result of the metabolism of livestock gradually decreasing, and thus the state determination unit 341 determines that the livestock has dead (S40). Note that, although an example in which the death of livestock is determined in step S40 in a case in which it is determined whether or not a change in the temperature difference ΔT is zero in step S18 and a change in the temperature difference ΔT is zero is shown, the state determination unit 341 may further determine a hyposthenia state which is a preliminary stage of death. In a case of determining hyposthenia, a threshold value X for hyposthenia determination is provided in addition to determining whether the temperature difference ΔT is zero, the state determination unit 341 may determine that the state of livestock is in the hyposthenia state in a case in which the temperature difference ΔT is equal to or greater than zero and equal to or less than the threshold value X for hyposthenia determination. On the other hand, in a case in which the time differentiated value dΔT/dt of the temperature difference ΔT is equal to or greater than the threshold value Δ (No in S20), since the temperature difference ΔT becomes relatively abruptly zero, the state determination unit 341 determines that the signal transmission device 10A has fallen out of livestock (S38).

In step S16 described above, in a case in which the voltage integrated value ∫V is not zero (No in S16), the state determination unit 341 determines whether or not the voltage integrated value ∫V is less than a threshold value α (S22). The threshold value a can be set to an appropriate value as a threshold value for determining that the activity amount of livestock is reduced as compared with the normal level. In a case in which the voltage integrated value ∫V is less than the threshold value α (Yes in S22), the state determination unit 341 determines whether or not the temperature difference ΔT is stable on the basis of the history of a change in the accumulated temperature difference ΔT (S24).

In a case in which the temperature difference ΔT is not zero and is stable (Yes in S24), since the metabolism of livestock is normal but the activity amount is small, the state determination unit 341 determines that the livestock is injured (S44). On the other hand, in a case in which the temperature difference ΔT is not stable (No in S24), the state determination unit 341 determines whether or not the temperature difference ΔT tends to increase on the basis of the history of a change in the accumulated temperature difference ΔT (S26). In a case in which the temperature difference ΔT tends to increase (Yes in S26), since the livestock generates heat and the activity amount decreases, the state determination unit 341 determines that the livestock is sick (S46).

In step S22 described above, the voltage integrated value ∫V is equal to or greater than the threshold value α (No in S22), the state determination unit 341 determines whether or not the voltage integrated value ∫V exceeds a threshold value β (S28). The threshold value β can be set to an appropriate value as a threshold value for determining that the activity amount of livestock largely increases as compared to the normal level. In a case in which the voltage integrated value ∫V exceeds the threshold value β (Yes in S28), the state determination unit 341 determines whether or not the temperature difference ΔT tends to increase on the basis of the history of a change in the accumulated temperature difference ΔT (S30). In a case in which the temperature difference ΔT tends to increase (Yes in S30), the state determination unit 341 determines that the livestock is in an esthetic state (S48).

In step S28 described above, in a case in which the voltage integrated value ∫V is equal to or less than the threshold value θ (No in S28), the state determination unit 341 determines whether or not the voltage integrated value ∫V exceeds a threshold value γ (S32). The threshold value γ can be set to an appropriate value as the threshold value γ for determining that the activity amount of livestock is not largely increased as compared with the normal level, but is somewhat increased. In a case in which the voltage integrated value ∫V exceeds the threshold values γ (Yes in S32), the state determination unit 341 determines whether or not the temperature difference ΔT tends to decrease on the basis of the history of a change in the accumulated temperature difference ΔT (S34). In a case in which the temperature difference ΔT tends to decrease (Yes in S34), the state determination unit 341 determines that the live stock is in the childbirth state (S50).

In a case in which the temperature difference ΔT is not zero (No in S18) in step S18, in a case in which the temperature difference ΔT does not tend to increase (No in S26) in step S26, in a case in which the temperature difference ΔT does not tend to increase (No in S30) in step S30, and in a case in which the voltage integrated value ∫V is equal to or less than the threshold value γ (No in S32) in step S32, the state determination unit 341 determines that there is no abnormality in the livestock (S42).

As above, an example of the state determination processing has been described, and the information processing server 30 can determine the biological information of livestock on the basis of the information related to a first temperature T1, the information related to a second temperature T2, and the vibration information included in a signal transmitted from the signal transmission device 10A attached to each head of livestock. Therefore, the breeder 9A or the veterinarian 9B accesses the information processing server 30 via the information terminals 9, thereby ascertaining the state of individual livestock. As a result, diagnosis, treatment, or the like of livestock can be timely performed when necessary.

Moreover, the information processing server 30 can detect a fall of the signal transmission device 10A on the basis of the information related to a first temperature T1, the information related to a second temperature T2, and the vibration information included in a transmission signal transmitted from the signal transmission device 10A attached to each head of livestock. In this case, since the transmission of a signal from the signal transmission device 10A can be continued even in a falling state, the existence range of the signal transmission device 10A falling can be narrowed on the basis of the identification information of the signal relay device 10B, and the signal transmission device 10A is easy to be found.

Not that the state determination processing show in FIG. 36 is merely an example, and the control unit 320 in the information processing device 30 may perform another appropriate state determination processing. For example, in a case in which the signal transmission device 10A includes a sensor other than the temperature sensor and the vibration sensor 335, the state determination unit 341 in the information processing device 30 may execute desirable state determination processing using information related to a measurement value detected by the sensor. In addition, the state determination unit 341 or the learning control unit 355 in the information processing device 30 may execute determination processing of the state of livestock using a learning algorithm such as artificial intelligent (AI) processing or machine learning, for example, with the vibration information, temperature information, estrus history, or the like as an input and the biological information including the state information of livestock and the activity amount of the livestock as an output.

### 6. SUMMARY

As described above, in the information processing system 1 according to the present embodiment, the breeder-side information terminal 50 and the veterinarian-side information terminal 70 can share digitized individual information and breeding information of the livestock 3, and various pieces of information of medication history, injury/disease history, milk component analysis result, and the like. In addition, estrus information related to breeding information and some information of pregnancy diagnosis, sex identification, childbirth examination, and the like can be easily accumulated by being selected on an input screen.

Moreover, since the activity amount and the like of the livestock 3, and a transition of the state quantity showing a characteristic change in the estrus period are displayed in the display of the breeding information, a breeder can determine the estrus of the livestock 3 while overlooking the transition of the state quantity. In addition, a breeder performs an operation input of causing a notification of estrus contact to be transmitted to a veterinarian, and thereby the notification is sent to the veterinarian and information of an estrus date is automatically registered in the database storage unit 330. Therefore, a breeder can promptly notify a veterinarian of the estrus of the livestock 3 even at night and can be prevented from forgetting to perform a notification. Moreover, a veterinarian can set up a schedule for an estrus diagnosis beforehand.

In addition, when a transition of the state quantity is displayed, a next predicted estrus date, and a reference transition and, furthermore, a predicted transition of the state quantity are displayed together, and thereby a breeder can easily predict a next estrus date, and it is possible to prevent fertilization timing from being missed by preparing for fertilization in advance.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

For example, in the information processing system 1 according to the present embodiment, a display image of a transition of the state quantity included in breeding information is merely an example, and various modifications can be made. For example, transition of measurement data and reference transition may be vertically or horizontally arranged side by side.

In addition, in the information processing system 1 according to the present embodiment, the flowchart of state determination processing executed by the information processing server 30 is merely an example, and various modifications can be made. For example, each step may be replaced before and after.

In the information processing system 1 according to the present embodiment, the information processing server 30 has performed state determination processing and control of display information of the breeder-side information terminal 50, but the technology of the present disclosure is not limited to such examples. For example, the information processing server 30 may be omitted and the breeder-side information terminal 50 may have the function of the information processing server 30 described above.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1)

An information processing device including:

a display information control unit configured to cause a transition of a state quantity showing a characteristic change in an estrus period of livestock to be displayed on a display unit of a breeder-side information terminal; and

a notification control unit configured to execute, in accordance with a notification request to cause estrus of the livestock to be notified to a veterinarian-side information terminal on a basis of an operation of the breeder-side information terminal, both notification processing to the veterinarian-side information terminal and registration processing of registering time information indicating a time at which the notification request is made in a database as estrus history of the livestock.

(2)

The information processing device according to (1),

in which the display information control unit causes a transition of the state quantity and a predicted estrus date to be displayed together.

(3)

The information processing device according to (1) or (2),

in which the display information control unit causes a transition of the state quantity and a last estrus date to be displayed together.

(4)

The information processing device according to any one of (1) to (3),

in which the display information control unit causes a transition of the state quantity and a reference transition of the state quantity to be displayed together.

(5)

The information processing device according to (4),

in which the reference transition is a transition of the state quantity in a last estrus cycle of each head of the livestock.

(6)

The information processing device according to (4),

in which the reference transition is an average transition of state quantities in past estrus cycles of each head of the livestock.

(7)

The information processing device according to (4),

in which the reference transition is an average transition of the state quantities in estrus cycles of a plurality of heads of livestock of a same type.

(8)

The information processing device according to any one of (4) to (7),

in which the display information control unit sets a point of time before at least a period of estrus cycle of the livestock from a present time as a base point, and causes a transition of the state quantity from the base point to the present time and a reference transition of the state quantity to be displayed together.

(9)

The information processing device according to any one of (1) to (8),

in which the display information control unit causes a transition of the state quantity and a predicted transition of the state quantity of each head of the livestock after a present time to be displayed together.

(10)

The information processing device according to any one of (1) to (9),

in which the display information control unit causes a transition of the state quantity and a predicted estrus duration time to be displayed together.

(11)

The information processing device according to any one of (1) to (10),

in which the display information control unit causes a transition of the state quantity and a predicted estrus intensity to be displayed together.

(12)

The information processing device according to any one of (4) to (11), including:

a learning control unit configured to execute learning processing of a reference transition of the state quantity on a basis of information of estrus history of each head of the livestock and information of history of the state quantity of each head of the livestock that are registered by the notification control unit.

(13)

The information processing device according to (12),

in which the display information control unit causes a reference transition of the state quantity updated by learning processing of the learning control unit to be displayed on the display unit of the breeder-side information terminal in conjunction with a transition of the state quantity.

(14)

The information processing device according to any one of (1) to (13),

in which the display information control unit causes a transition of the state quantity and a graphical user interface part for the notification request to be displayed together.

(15)

The information processing device according to (14),

in which the display information control unit causes information indicating livestock scheduled for estrus to be displayed on the display unit, and

the display unit is caused to display a transition of the state quantity of livestock corresponding to a selection operation of the displayed information indicating livestock scheduled for estrus, in accordance with the selection operation.

(16)

The information processing device according to any one of (1) to (15),

in which the display information control unit causes a transition of the state quantity to be displayed on the display unit of the veterinarian-side information terminal when a display request is received from the veterinarian-side information terminal.

(17)

The information processing device according to any one of (2) to (16),

in which the display information control unit predicts the predicted estrus date on a basis of history of information of at least one of an estrus date, a fertilization date, an estrus intensity, and an estrus duration time of each head of the livestock.

(18)

The information processing device according to any one of (1) to (17), including:

a state determination unit configured to estimate estrus of the livestock on a basis of a transition of an activity amount of the livestock serving as the state quantity, and a metabolic rate of the livestock.

(19)

An information processing system including:

a breeder-side information terminal configured to display a transition of a state quantity showing a characteristic change in an estrus period of livestock;

an information processing device that includes a notification control unit configured to execute, in accordance with a notification request to cause estrus of the livestock to be notified to a veterinarian-side information terminal on a basis of an operation of the breeder-side information terminal, both notification processing to the veterinarian-side information terminal and registration processing of registering time information indicating a time at which the notification request is made in a database as estrus history of the livestock; and

the veterinarian-side information terminal configured to receive the notification and perform a notification operation.

(20)

An information processing method including: causing a transition of a state quantity showing a characteristic change in an estrus period of livestock to be displayed on a display unit of a breeder-side information terminal of a breeder of the livestock; and

executing, in accordance with a notification request to cause estrus of the livestock to be notified to a veterinarian-side information terminal on a basis of an operation of the breeder-side information terminal, both notification processing to the veterinarian-side information terminal and registration processing of registering time information indicating a time at which the notification request is made in a database as estrus history of the livestock.

### REFERENCE SIGNS LIST

1 information processing system
10A signal transmission device
10B signal relay device
20 communication network connection device
30 information processing device (information processing server)
50 breeder-side information terminal
70 veterinarian-side information terminal
310 communication network connection unit
320 control unit
325 database control unit
330 database storage unit
340 signal processing unit
341 state determination unit
343 vibration generated electric power integration unit
345 temperature difference calculation unit
350 information control unit
351 display information control unit
353 notification control unit
355 learning control unit

1. An information processing device comprising:
a display information control unit configured to cause a transition of a state quantity showing a characteristic change in an estrus period of livestock to be displayed on a display unit of a breeder-side information terminal; and
a notification control unit configured to execute, in accordance with a notification request to cause estrus of the livestock to be notified to a veterinarian-side information terminal on a basis of an operation of the breeder-side information terminal, both notification processing to the veterinarian-side information terminal and registration processing of registering time information indicating a time at which the notification request is made in a database as estrus history of the livestock.

2. The information processing device according to claim 1,
wherein the display information control unit causes a transition of the state quantity and a predicted estrus date to be displayed together.

3. The information processing device according to claim 1,
wherein the display information control unit causes a transition of the state quantity and a last estrus date to be displayed together.

4. The information processing device according to claim 1,
wherein the display information control unit causes a transition of the state quantity and a reference transition of the state quantity to be displayed together.

5. The information processing device according to claim 4,
wherein the reference transition is a transition of the state quantity in a last estrus cycle of each head of the livestock.

6. The information processing device according to claim 4,
wherein the reference transition is an average transition of state quantities in past estrus cycles of each head of the livestock.

7. The information processing device according to claim 4,
wherein the reference transition is an average transition of the state quantities in estrus cycles of a plurality of heads of livestock of a same type.

8. The information processing device according to claim 4,
wherein the display information control unit sets a point of time before at least a period of estrus cycle of the livestock from a present time as a base point, and causes a transition of the state quantity from the base point to the present time and a reference transition of the state quantity to be displayed together.

9. The information processing device according to claim 1,
wherein the display information control unit causes a transition of the state quantity and a predicted transition of the state quantity of each head of the livestock after a present time to be displayed together.

10. The information processing device according to claim 1,
wherein the display information control unit causes a transition of the state quantity and a predicted estrus duration time to be displayed together.

11. The information processing device according to claim 1,
wherein the display information control unit causes a transition of the state quantity and a predicted estrus intensity to be displayed together.

12. The information processing device according to claim 4, comprising:
a learning control unit configured to execute learning processing of a reference transition of the state quantity on a basis of information of estrus history of each head of the livestock and information of history of the state quantity of each head of the livestock that are registered by the notification control unit.

13. The information processing device according to claim 12,
wherein the display information control unit causes a reference transition of the state quantity updated by learning processing of the learning control unit to be displayed on the display unit of the breeder-side information terminal in conjunction with a transition of the state quantity.

14. The information processing device according to claim 1,
wherein the display information control unit causes a transition of the state quantity and a graphical user interface part for the notification request to be displayed together.

15. The information processing device according to claim 14,
wherein the display information control unit causes information indicating livestock scheduled for estrus to be displayed on the display unit, and
the display unit is caused to display a transition of the state quantity of livestock corresponding to a selection operation of the displayed information indicating livestock scheduled for estrus, in accordance with the selection operation.

16. The information processing device according to claim 1,
wherein the display information control unit causes a transition of the state quantity to be displayed on the display unit of the veterinarian-side information terminal when a display request is received from the veterinarian-side information terminal.

17. The information processing device according to claim 2,
wherein the display information control unit predicts the predicted estrus date on a basis of history of information of at least one of an estrus date, a fertilization date, an estrus intensity, and an estrus duration time of each head of the livestock.

18. The information processing device according to claim 1, comprising:
a state determination unit configured to estimate estrus of the livestock on a basis of a transition of an activity amount of the livestock serving as the state quantity, and a metabolic rate of the livestock.

19. An information processing system comprising:
a breeder-side information terminal configured to display a transition of a state quantity showing a characteristic change in an estrus period of livestock;
an information processing device that includes a notification control unit configured to execute, in accordance with a notification request to cause estrus of the livestock to be notified to a veterinarian-side information terminal on a basis of an operation of the breeder-side information terminal, both notification processing to the veterinarian-side information terminal and registration processing of registering time information indicating a time at which the notification request is made in a database as estrus history of the livestock; and
the veterinarian-side information terminal configured to receive the notification and perform a notification operation.

20. An information processing method comprising:
causing a transition of a state quantity showing a characteristic change in an estrus period of livestock to be displayed on a display unit of a breeder-side information terminal of a breeder of the livestock; and
executing, in accordance with a notification request to cause estrus of the livestock to be notified to a veterinarian-side information terminal on a basis of an operation of the breeder-side information terminal, both notification processing to the veterinarian-side information terminal and registration processing of registering time information indicating a time at which the notification request is made in a database as estrus history of the livestock.
